(19) United States
(12) Patent Application Publication
(10) Pub. No.: US 2022/0401378 A1
(43) Pub. Date: Dec. 22, 2022

(54) COMPOSITIONS COMPRISING FUNCTIONAL FRAGRANCES AND CANNABIS-DERIVED COMPOUNDS

(71) Applicant: THIS WORKS PRODUCTS LIMITED, Wimbledon (GB)

(72) Inventor: Anna PERSAUD, Wimbledon (GB)

(21) Appl. No.: 17/642,081

(22) PCT Filed: Mar. 8, 2021
(86) PCT No.: PCT/IB2021/051917

§ 371 Date: Mar. 10, 2022

Related U.S. Application Data
(60) Provisional application No. 62/986,929, filed on Mar. 9, 2020.

(51) Int. Cl. A61K 31/05; A61K 31/07; A61K 36/9068; A61K 36/889; A61K 36/53; A61K 36/54; A61K 36/899; A61K 36/28; A61K 36/87; A61K 36/67; A61K 36/61; A61K 33/06

(52) U.S. Cl. CPC A61K 31/05; A61K 31/07; A61K 36/9068; A61K 36/889; A61K 36/53; A61K 36/54; A61K 36/899; A61K 36/28; A61K 36/87; A61K 36/67; A61K 36/61; A61K 33/06

(57) ABSTRACT

The present technology generally relates to compositions comprising at least one purified cannabinoid or a derivative thereof, and at least one functional fragrance. The present technology also generally relates to methods of using the compositions for, inter alia, improving sleep, for treating insomnia and/or related sleep disorders.

### CROSS-REFERENCE TO RELATED APPLICATION

This application claims the benefit of and priority to U.S. provisional patent application No. 62/986,929, filed on Mar. 9, 2020; the content of which is herein incorporated in entirety by reference.

### FIELD

The present disclosure is directed to compositions comprising a functional fragrance and a cannabis-derived compound. The present disclosure is also directed to methods of using such compositions for, inter alia, improving sleep.

### BACKGROUND

Insomnia and a host of related sleep disorders described herein afflict a significant, but largely unquantified, number of people at some point during their lives. Between 10% and 30% of adults have insomnia at any given point in time and up to half of people have insomnia at a point in each year. About 6% of people have insomnia that is not due to another problem and lasts for more than a month. People over the age of 65 are affected more often than younger people. Females are more often affected than males.

Insomnia, also known as sleeplessness, is a sleep disorder wherein people have trouble sleeping. They may have difficulty falling asleep or staying asleep if desired. Insomnia is typically followed by daytime sleepiness, low energy, irritability, and a depressed mood. It may result in an increased risk of motor vehicle collisions, as well as problems focusing and learning. Insomnia can be short term, lasting for days or weeks, or long term, lasting for more than a month.

Insomnia can occur independently or because of another problem. Conditions that can result in insomnia include, by way of example and not limitation, psychological stress, chronic pain, heart failure, hyperthyroidism, heartburn, restless leg syndrome, menopause, certain medications, and drugs such as caffeine, nicotine, and alcohol. Other risk factors include working night shifts and sleep apnea. Diagnosis is based on sleep habits and an examination to look for underlying causes. A sleep study may be done to look for underlying sleep disorders. Screening may be done with two questions: “do you have trouble sleeping?” and “do you have difficulty falling or staying asleep?”.

Sleep hygiene and lifestyle changes are typically the first treatment for insomnia Sleep hygiene includes a consistent bedtime, exposure to sunlight, a quiet and dark room, and regular exercise. Cognitive behavioral therapy may be added to this. While sleeping pills may help, they may be associated with injuries, dementia, and addiction, among other side effects. Medications are not recommended for more than four or five weeks. The effectiveness and safety of alternative medicine is to date unclear, however natural aromatherapy-based products such as pillow sprays and topical sleep aids are used for improving sleep quality and aiding restless sleep.

There is a need in the art for alternative methods of improving sleep quality and diminishing effects of insomnia and sleep disorders.

### SUMMARY

The present disclosure provides compositions and methods of using the compositions including a plurality of naturally occurring, synthetic or semisynthetic compounds for treatment of sleep disorders, including various forms of insomnia in a subject in need thereof. In particular, the present disclosure is directed toward compositions, methods of making and using the compositions for providing improved sleep in a subject. The compositions comprise a combination of a functional fragrance and compounds found in cannabis.

In a broad aspect, there is provided a composition for providing improved sleep in a subject, the composition comprising at least one purified cannabinoid or a derivative thereof, and at least one functional fragrance. In some embodiments, the at least one purified cannabinoid is cannabidiol (CBD). The at least one purified cannabinoid may be present in the composition in an amount ranging from about 1 wt % to about 5 wt %, or from about 3 wt % to about 5 wt %, or in about 3 wt %, about 4 wt %, or about 5 wt % of the composition. The purified cannabinoid may be isolated from Cannabis or synthetically derived.

In some embodiments, the at least one functional fragrance comprises one or more essential oil such as, without limitation, lavender, vetivert, chamomile, patchouli, ho wood, grapeseed, marjoram, black pepper, clove and/or coconut oil. The functional fragrance may also comprise at least one purified terpene such as, without limitation, linalool, linalool oxide, limonene, geraniol, citronellol, eugenol, citral, farnesol, bakuchiol, derivatives thereof, and combinations thereof. In some embodiments, the functional fragrance comprises one or more of the following: Linalool, Linalyl Acetate, Vetiverols, Ocimene, Caryophyllene, Terpentine-4-ol, beta-Pinene, d-Limonene, 1-Octen-3-yl acetate, Geraniol, derivatives thereof, and combinations thereof. The functional fragrance may comprise odorants extracted or isolated from natural sources, synthetic odorants, and combinations thereof.

In some embodiments, the composition of the present technology or at least a portion thereof is encapsulated to allow for delayed or continuous release of at least a portion of the functional fragrance. In some embodiments, the encapsulate is broken via friction to release at least a portion of the functional fragrance. In an embodiment, the encapsulate is comprised essentially of naturally derived plant materials and the encapsulate comprises one or more of sodium alginate, calcium chloride, Riboflavin 5-Phosphate, Genipin, and combinations thereof. In an embodiment, the encapsulate is from about 5 microns to about 25 microns in size.

Compositions of the present technology may be administered topically, transdermally, nasally, or by inhalation. In some embodiments, the composition is in the form of an ointment, a cream, a suspension, a liniment, a balm, a serum, a lotion, a paste, a gel, a spray, an aerosol, a foam, an oil, a liquid, a powder, a patch, a matrix, or a wax. In some instances, the composition is in a sprayable form. For example, the composition may be dispensed in the form of an aerosol spray. In certain embodiments, the composition is in the form of a pillow spray and is administered by spraying onto a surface adjacent or near to a subject's head before the subject's head is placed adjacent or near thereto, such as on the subject's pillow or bed linens.

In another broad aspect, there is provided a method for improving sleep in a subject in need thereof, the method comprising administering to the subject an effective amount of the composition described herein, such that the subject has improved sleep. The composition may improve a subject's sleep by providing improvement in one or more of the following, compared to sleep without administration of the composition: total sleep time; sleep efficiency; sleep latency; amount of rapid eye movement (REM) sleep; latency of REM sleep; number of awakenings; sleep latency after awakening; amount of deep sleep; subjective feeling of waking up refreshed or rested; and combinations thereof.

Another aspect of the present disclosure is directed to a method of treating insomnia and related sleep disorders in a subject in need thereof, the method comprising administering to the subject an effective amount of the composition described herein, such that insomnia or a related sleep disorder is treated in the subject.

Another aspect of the present disclosure is directed to a method for the treatment of muscle pain in a subject in need thereof, the method comprising administering to the subject an effective amount of the composition described herein, such that muscle pain is treated in the subject.

Another aspect of the present disclosure is directed to a method for moisturising and/or hydrating the skin of a subject in need thereof, the method comprising administering to the subject an effective amount of the composition described herein, such that the skin of the subject is moisturized and/or hydrated. Another aspect of the present disclosure is directed to a method for reducing the appearance of wrinkles on a skin portion of a subject in need thereof, the method comprising administering to the subject an effective amount of the composition described herein, such that the appearance of wrinkles is reduced.

It is further contemplated that various kits and dosage devices will be provided for use with the compositions of the disclosure.

Other aspects and features of the present disclosure will become apparent to those ordinarily skilled in the art upon review of the following description of specific embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the technology and to show more clearly how it may be carried into effect, reference will now be made by way of example to the accompanying drawings, which illustrate aspects and features according to non-limiting embodiments of the present technology.

FIG. 1 is a histogram showing intensity ratings within the scanner for the indicated fragrances. Significant effects of odor were observed, but no effect of repetition. The error bars indicate the 95% confidence interval.

FIG. 2 shows comparison of the neural activation following presentation of the DeepSleep®, Oriental® or Control (PEA) odor. The activations vs baseline were adjusted for intensity effects and presented on a standardized template with a height threshold of p<0.001, uncorrected.

FIG. 3 shows neural activation following presentation of DeepSleep®, adjusted for intensity effects and presented on a standardized template. The threshold level is liberally set to p<0.01, for visualization purpose.

FIG. 4 shows neural activation following presentation of Oriental®, adjusted for intensity effects and presented on a standardized template. The threshold level is liberally set to p<0.01, for visualization purpose.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “purified” means extracted, isolated, and/or separated from other compounds, formulations, compositions, matter, and/or mass resulting in a greater than 60% purity.

In some embodiments a “purified” cannabinoid (or “purified” terpene) is greater than about 70% pure, greater than 75% pure, greater than about 80% pure, greater than 85% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure. Within the context of the present disclosure, where a compound comprises stereogenic centers, the term “purified” includes enantiomerically pure compositions and also mixtures of enantiomers or isomers.

Also within the context of the present disclosure, purified compounds may be purposely formulated with other compounds at various levels of purity. Provided that the ingredients used for purposeful formulation are purified prior to the said purposeful formulation, the act of subsequently formulating them does render them not “purified” within the context of an ingredient list.

In an embodiment, the term “purified” may refer to a cannabinoid that s separated from plant flatter from which it was derived.

In an embodiment, the term “purified” may refer to a terpene that is separated from plant matter from which it was derived.

As used herein, the term “Cannabis” refers to the genus of flowering plants in the family Cannabaceae. Three species may be recognized as being part of the Cannabis genus, namely: Cannabis sativa, Cannabis indica, and Cannabis ruderalis. The expressions “Cannabis sativa” and “C. sativa” are used herein interchangeably. The term “strain” as used herein refers to different varieties of the plant genus Cannabis. For example, the term “strain” can refer to different pure or hybrid varieties of cannabis plants. In some instances, the cannabis strain of the present technology can by a hybrid of two strains, for example, a hybrid between C. sativa and C. indica. Different cannabis strains often exhibit distinct chemical compositions with characteristic levels of cannabinoids and terpenes, as well as other components. Differing cannabinoid and terpene profiles associated with different cannabis strains can be useful for the treatment of different diseases, or for treating different subjects with the same disease.

The expression “cannabis oil” as used herein refers to a mixture of compounds obtained from the extraction of cannabis plants. Such compounds include, but are not limited to, cannabinoids, terpenes, terpenoids, and other compounds found in the cannabis plant. The exact composition of cannabis oil depends on the strain of cannabis that is used for extraction, the efficiency and process of the extraction itself, and any additives that might be incorporated to alter the palatability or improve administration and/or bioavailability of the cannabis oil.

The term “eluate” as used herein refers to a solution that is collected after contacting a plant material, such as raw cannabis plant material, with an extraction solvent. The eluate can contain dissolved cannabinoids as well as other compounds. The term “filtrate” refers to a solution that has passed through a membrane or strainer of variable porousness or permeability to remove either particulate matter or unwanted compounds. As used herein, the term “distillate” refers to a solution that has been concentrated by any known means of evaporation or distillation. In some embodiments of the present technology, the filtrate is evaporated to form the distillate. The term “extract” as used herein refers to a solution that has been purged or dehydrated to remove residual solvent. In some embodiments of the present technology, the extract is formed by purging or dehydrating the distillate using any known means in the art. As used herein, the term “isolate” refers to a chemical substance that has been separated from foreign or contaminating substances. Pure results of a successful purification process are termed isolate. In some embodiments of the present technology, the isolate is refined distillate.

As used herein, the term “cannabinoid” refers to a chemical compound belonging to a class of secondary compounds commonly found in plants of genus cannabis, but also encompasses synthetic and semi-synthetic cannabinoids.

The most notable cannabinoid is tetrahydrocannabinol (THC), the primary psychoactive compound in cannabis Cannabidiol (CBD) is another cannabinoid that is a major constituent of the phytocannabinoids. There are at least 113 different cannabinoids isolated from cannabis, exhibiting varied effects.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

As used herein, the expression “cannabinoid concentrate” refers to products made from the cannabis plant that have been processed to keep only the most desirable plant compounds (primarily the cannabinoids), while removing excess plant material and other impurities. As used herein, the expression “cannabinoid concentrate” includes one or more of cannabinoid distillate and cannabinoid isolate (e.g., crystalline CBD).

As used herein, the term “endocannabinoid” refers to the biological system composed of endocannabinoids, which are endogenous lipid-based retrograde neurotransmitters that bind to cannabinoid receptors, and cannabinoid receptor proteins that are expressed throughout the vertebrate central nervous system (including the brain) and peripheral nervous system.

The expression “cannabinoid receptor” refers hereinafter to a class of cell membrane receptors under the G protein-coupled receptor superfamily. There are currently two known subtypes of cannabinoid receptors, termed CB₁ and CB₂. The CB₁ receptor is expressed in the brain, the lungs, the liver, the kidneys, and throughout the body. The CB₂ receptor is expressed mainly in the immune system and in hematopoietic cells. The expression “Cannabinoid receptor type 1 (CB₁)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC. The expression “Cannabinoid receptor type 2 (CB₂)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids).

As used herein, the expression “effective amount” or “therapeutically effective amount” refers to the amount of components of the compositions of the present disclosure which are effective for producing some desired therapeutic effect as defined herein at a reasonable benefit/risk ratio applicable to any treatment.

In one embodiment, the present technology relates to compositions comprising at least one cannabinoid compound, e.g., a purified cannabinoid, and at least one functional fragrance. In some implementations of this embodiment, the compositions of the present disclosure are useful in providing improved sleep in a subject (e.g., a subject in need of such treatment).

1) Purified Cannabinoid

In some embodiments the “purified” cannabinoid present in the compositions of the present technology is greater than about 60% pure, greater than about 70% pure, greater than about 80% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure.

In some embodiments, the cannabinoid present in the compositions of the present technology is selected from one or more of: Cannabigerol-type (CBG): cannabigerol ((E)-CBG C-5), cannabigerol monomethyl ether ((E)-CBGM C-5A), Cannabinerolsäure A ((Z)-CBGA C-5A), Cannabigerovarin (((e)-BGV C-3), Cannabigerolsäure A(e)-CBGA C-5A), A Cannabigerolsäure monomethyl ether ((e)-CBGAM C-5A), Cannabigerovarinsäure A ((e)-CBGVA-C3A); Cannabichromene-type (CBC): cannabichromene (CBC-C5), Cannabichromensäure A (CBCA C-5A), Cannabichromevarin (CBCVC-3), Cannabichromevarinsäure A (CBCVA-C3A); Cannabidiol-type (CBD), cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3); Cannabinodiol-like (CBND): Cannabinodiol (CBND C-5), Cannabinodivarin (CBND C-3); Tetrahydrocannabinol-like (THC): Δ⁹-tetrahydrocannabinol (Δ⁹-THC-C5), Δ⁹-tetrahydrocannabinol-C4 (Δ⁹-THC-C4), Δ⁹-tetrahydrocannabivarin (Δ⁹-THCV-C3), Δ⁹-Tetrahydrocannabiorcol (Δ⁹-THCO C-1), Δ⁹-Tetrahydrocannabinolsäure (Δ⁹ THCA-C-5A), Δ⁹-Tetrahydrocannabinolsäure B (Δ⁹ THCA-C-5B), Δ⁹-Tetrahydrocannabinolsäure-C4 (4⁹ THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1 A and/or B), (−)-Δ⁸-trans-(6aR,10aR)-8-tetrahydrocannabinol (Δ⁸-THC-C 5), (−)-Δ⁸-trans-(6aR,10aR)-Tetrahydrocannabinolsäure A (Δ⁸-THCA-C 5A); (−)-(6a S, 10a R)-Δ⁹-tetrahydrocannabinol ((−)-cis-Δ⁹-THC-C5); Cannabinol-type (CBN): Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5) Cannabitriol-type (CBT): (−)-(9R,10R)-trans-Cannabitriol ((−)-trans-CBT-C5), (+)-(9S,10S)-Cannabitriol ((+)-trans-CBT C-5), (±)-(9R,10S/9S, 10R)-Cannabitriol ((f)-cis-CBT-C5), (−)-(9R,10R)-trans [10-0-thyl-cannabitriol] ((−)-trans-CBT-OEt-C5), (±)-(9R,10R/9S, 10 S)-Cannabitriol-C3 ((±)-trans-CBT-C3), 8,9-dihydroxy-46a (10a) tetrahydrocannabinol (8,9-di-OH-CBT-C5), cannabidiolic A (CBDA C-59-OH-CBT-C5 ester), (−)-(6aR,9S, 10S, 10aR)-9,10-dihydroxy-hexahydrocannabinol, Cannabiripsol Cannabiripsol-C5, (−)-6a,7,10a-trihydroxy-Δ⁹-tetrahydrocannabinol ((−)-Cannabitetrol), 10-oxo-46a (10a) tetrahydrocannabinol (OTHC); Cannabielsoin-like (CBE): (5aS,6S, 9R, 9aR)—C5-Cannabielsoin (CBEC-5), (5aS,6S, 9R, 9aR)-C3-Cannabielsoin (CBE C-3), (5aS,6S, 9R, 9aR)-Cannabielsoinsäure A (CBEA-C5 A), (5aS,6S, 9R, 9aR)-Cannabielsoinsäure B (CBEA-C5 B), (5aS,6S, 9R, 9aR)-C3 Cannabielsoinsäure B (CBEA-C3 B), Cannabiglendol-C3 (OH-iso-HHCV C-3), Dehydrocannabifuran (DCBF C-5), Cannabifuran (CBF-C5); Isocannabinoide: (−)-Δ⁷-trans-(1R,3R, 6R)Isotetrahydrocannabinol, (±)-Δ⁷-1,2-cis-(1R,3R, 6S/1S, 3S, 6R)-Isotetrahydrocannabivarin, (+47-trans-(1R,3R, 6R)-Isotetrahydrocannabivarin; Cannabicyclol-like (CBL): (±)-(1aS,3aR, 8bR, 8Cr-cannabicyclol (CBL-C), (±)-(1aS,3aR, 8bR, 8Cr-Cannabicyclolsäure A (CBLA-C5A) (±)-(1aS,3aR, 8bR, 8Cr-Cannabicyclovarin (CBLV C-3); Cannabicitran-type (CBT): Cannabicitran (CBT-C5); Cannabichromanon-like (CBCN) Cannabichromanon (CBCN C-5), Cannabichromanon-C3 (CBCN C-3), Cannabicoumaronon (CBCON C-5). In addition to the above cannabinoids, the carboxylic acids which are biosynthetic precursors of each are contemplated as cannabinoids which may be present in the compositions of the present technology.

In some implementations of these embodiments, the compositions of the present technology comprise one or more of: CBD, THC, CBG, CBN, CBC, THCV, CBGA, CGCA, CBCA, THCA and CBDA.

In some implementations of these embodiments, the compositions of the present technology may comprise a predominant cannabinoid and minimal or trace amounts of other cannabinoids. For example, the composition of the present technology may comprise CBD as a predominant cannabinoid and trace amounts or minimal amounts of THC. As used herein, the expression “CBD:THC ratio” or “THC:CBD ratio” refers to the amount of CBD and THC that is comprised in any the compositions of the present technology. THC dominant options (0:1) will provide varying degrees of psychoactivity. CBD dominant ratios of 100:1, 25:1 or 1:0 offer little or no psychoactivity.

In some embodiments, the compositions of the present technology comprise cannabinoid in an amount ranging from between about 0.001 wt % to about 99.999 wt %, between about 0.001 wt % and about 90 wt %, between about 0.01 wt % and about 90 wt %, between about 0.1% wt % and about 90 wt %, between about 0.001 wt % to about 75 wt %, between about 0.001% and about 75 wt %, between about 0.01 wt % and about 75 wt %, between about 0.1% wt % and about 75 wt %, between about 0.001 wt % to about 50 wt %, between about 0.001 wt % and about 50 wt %, between about 0.01 wt % and about 50 wt %, between about 0.1% wt % and about 50 wt %, between about 0.001 wt % to about 25 wt %, between about 0.001 wt % and about 25 wt %, between about 0.01 wt % and about 25 wt %, between about 0.1% wt % and about 25 wt %, between about 0.001 wt % and about 20 wt %, between about 0.5 wt % and about 10 wt %, between about 1 wt % and about 5 wt %, between about 2 wt % and about 5 wt %, between about 3 wt % and about 5 wt %, between about 1 wt % and about 3 wt %, between about 2 wt % and about 3 wt %, about 1 wt %, about 2 wt %, about 3 wt %, about 4 wt %, about 5 wt %, about 6 wt %, about 7 wt %, about 8 wt %, about 9 wt % or about 10 wt % of the total weight of the composition.

In some implementations, the cannabinoid is cannabidiol (CBD) or a derivative thereof selected from CBD, CBDV, CBDA and any combination thereof and wherein the CBD or a derivative thereof is selected from natural CBD or a derivative thereof produced in the body of humans and animals, CBD or a derivative thereof extracted from plants, synthetic CBD or a derivative thereof, and any combination thereof. In some such embodiments, the compositions of the present technology comprise CBD or a derivative thereof in an amount of about 1 wt %, about 2 wt %, about 3 wt %, about 5 wt %, about 6 wt %, about 7 wt %, about 8 wt %, about 9 wt % or about 10 wt % of the total weight of the composition.

In some embodiments, the compositions of the present technology comprise CBD in an amount of about 1 wt %, about 2 wt %, about 3 wt %, about 4 wt %, about 5 wt %, about 6 wt %, about 7 wt %, about 8 wt %, about 9 wt % or about 10 wt % of the total weight of the composition.

In various embodiments provided herein, percent refers to percent by weight. In one embodiment, the cannabis isolate or extract comprises about 95% or more of at least one desired cannabinoid. In one embodiment, the cannabis isolate or extract comprises about 90% or more of at least one desired cannabinoid. In one embodiment, the cannabis isolate or extract comprises about 80% or more of at least one desired cannabinoid. In one embodiment, the cannabis isolate or extract comprises about 70% or more of at least one desired cannabinoid. In one embodiment, the cannabis isolate or extract comprises about 60% or more of at least one desired cannabinoid. In one embodiment, the cannabis isolate or extract comprises about 50% or more of at least one desired cannabinoid. In one embodiment, the cannabis isolate or extract comprises about 40% or more of at least one desired cannabinoid. In one embodiment, the cannabis isolate or extract comprises about 30% or more of at least one desired cannabinoid. In one embodiment, the cannabis isolate or extract comprises about 20% or more of at least one desired cannabinoid. In one embodiment, the cannabis isolate or extract comprises about 10% or more of at least one desired cannabinoid. In one embodiment, the cannabis isolate or extract comprises about 5% or more of at least one desired cannabinoid. In one embodiment, the cannabis isolate or extract comprises about 1% or more of at least one desired cannabinoid. In one embodiment, the cannabis isolate or extract comprises about 0.1% or more of at least one desired cannabinoid. In one embodiment, the cannabis isolate or extract comprises about 0.01% or more of at least one desired cannabinoid. In one embodiment, the cannabis isolate or extract comprises about 0.001% or more of at least one desired cannabinoid. In one aspect, the cannabis isolate or extract is diluted to a desired cannabinoid concentration.

In some embodiments, the composition as defined herein is non-psychoactive and does not exhibit any psycho-effect on the subject.

Cannabinoids may be extracted from Cannabis plants or parts thereof using various solvents and technologies including, but not limited to, ethanol, butane, methane, heptane, carbon dioxide, ice, water, and steam. A cannabinoid of interest may be extracted from Cannabis plants bred to express a desired profile of the cannabinoid of interest for purity. In some implementations, the cannabinoid may be extracted using supercritical fluid (SFC) extraction and similar technologies. In some instances, the cannabinoid is crystalized. The process of crystallization involves placing the compound of interest in a liquid and then cooling or adding participants to the solution which would lower the solubility of the compound of interest so that it forms crystals. In this example, crystals are then separated from the liquid through filtration or centrifugation.

In some embodiments, the cannabinoid useful in the compositions of the present technology is a synthetic cannabinoid or is a biosynthetic cannabinoid.

In some implementations of these embodiments, the cannabinoid is cannabidiol (CBD) or a derivative thereof. Derivatives of cannabidiol that may be used in the compositions of the present technology include compounds that can be imagined to arise or actually be synthesized from a cannabidiol by replacement of one atom of its structure with another atom or with a group of atoms. An example of a derivative of CBD is cannabidiol dimethyl heptyl (CBD-DMH), including all isomeric forms thereof.

ii) Functional Fragrances

As used herein, the term “functional fragrance” refers to a compound that activates olfactory receptors leading to activation of neural circuitry in the brain promoting psychological or physical well-being. A functional fragrance may, for example, provide improved sleep, relaxation, calm, and/or reduced anxiety.

In some embodiments, a functional fragrance comprises one or more essential oil.

In some embodiments, a functional fragrance comprises one or more purified terpene.

In some embodiments, a functional fragrance comprises one or more essential oil and one or more terpene.

A wide range of odorants may be used in functional fragrances in the compositions of the present technology. A functional fragrance may include odorants extracted or isolated from natural sources, synthetic odorants, and combinations thereof.

As used herein, an “essential oil” refers to a natural oil (a concentrated hydrophobic liquid) typically obtained by distillation and having the characteristic fragrance of the plant or source from which it is extracted. Essential oils generally contain volatile chemical compounds that give plants their characteristic odors and are used especially in perfumes, flavoring, and for aromatherapy. Non-limiting examples of essential oils that may be used in compositions of the present technology include orange, lavender, peppermint, thyme, patchouli, oregano, lemon, grapefruit, bergamot, tea tree, ho wood, eucalyptus, mugwort, pennyroyal, chamomile, wormwood, marjoram, black pepper, clary sage, sage, clove, rosemary, cinnamon, lemongrass, ylang ylang, sandalwood, jasmine, rose, vetivert, frankincense, myrrh, helichrysum, cedarwood, spearmint, basil, and the like. In some embodiments, lavender, vetivert and chamomile are used in functional fragrances in the compositions of the present technology. In other embodiments, lavender, vetivert, chamomile and patchouli are used. In still other embodiments, lavender, vetivert, chamomile, patchouli and ho wood are used.

In some embodiments, marjoram, black pepper and clove oils are used in functional fragrances in the compositions of the present technology.

In some embodiments, the functional fragrance comprises one or more essential oil selected from lavender oil, vetivert oil, chamomile oil, patchouli oil, ho wood oil, grapeseed oil, coconut oil, and combinations thereof.

In some embodiments, the functional fragrance comprises one or more essential oil selected from lavender oil, vetivert oil, chamomile oil, patchouli oil, ho wood oil, grapeseed oil, coconut oil, marjoram oil, black pepper oil, clove oil and combinations thereof.

In some embodiments, the functional fragrance useful in the compositions of the present technology comprises Lavender oil. Lavender oil is an essential oil obtained by distillation from the flower spikes of certain species of lavender. Lavender oil has long been used as a perfume, for aromatherapy, and for skin applications for its relaxation, calming, anxiolytic, soothing and sedative effects. In some such embodiments, the composition of the present technology thus comprises CBD or a derivative thereof, and lavender oil, or an extract thereof.

In some embodiments, the functional fragrance useful in the compositions of the present technology comprises between about 0.005 wt % and about 25 wt % lavender oil, or between about 0.01 wt % and about 10 wt % lavender oil, or between about 0.025 wt % and about 5 wt % lavender oil, or between about 0.3 wt % and about 5 wt % lavender oil, or between about 1 wt % and about 10 wt % lavender oil.

In some embodiments, the functional fragrance useful in the compositions of the present technology comprises vetivert oil. Vetivert oil (Vetiver Essential Oil) is derived from the aromatic roots of the Vetiveria zizanioides (Linn) Nash. botanical. Due to its grounding, sensuous, and deeply calming scent, vetiver essential oil is also referred to as the “Oil of Tranquility” and “The Fragrance of the Soil.” Used in aromatherapy applications, vetiver essential oil is beneficial for soothing anxiety, insomnia, fatigue, depression, and inability to concentrate or remember. In some such embodiments, the composition of the present technology thus comprises CBD or a derivative thereof, and vetivert oil, or an extract thereof.

In some embodiments, the functional fragrance useful in the compositions of the present technology comprises between about 0.005 wt % and about 10 wt % vetiver oil, or between about 0.025 wt % and about 10 wt % vetiver oil, or between about 0.1 wt % and about 10 wt %, or between about 1 wt % and about 10 wt % vetivert oil, or between about 3.5 wt % and about 10 wt % vetivert oil

In some embodiments, the functional fragrance useful in the compositions of the present technology comprises chamomile oil. Chamomile essential oil has powerful anti-inflammatory and calming properties. The main benefits of chamomile oil are its ability to calm nerves, fight anxiety and depression. Its calming compounds serve as an emotional trigger to help reduce anxiety, boost mood and eliminate stress. In some such embodiments, the composition of the present technology thus comprises CBD or a derivative thereof, and chamomile oil, or an extract thereof.

In some embodiments, the functional fragrance useful in the compositions of the present technology comprises between about 0.005 wt % and about 10 wt % chamomile oil, or between about 0.05 wt % and about 5 wt % chamomile oil, or between about 0.025 wt % and about 5 wt % chamomile oil, or between about 0.1 wt % and about 5 wt % chamomile oil, or between about 1.9 wt % and about 5 wt % chamomile oil.

In some embodiments, the functional fragrance useful in the compositions of the present technology comprises patchouli oil. Patchouli oil is derived from the leaves of the highly-valued Patchouli plant, which belongs to a family of other well-known aromatic plants, including Lavender, Mint, and Sage. It has a warm, spicy, musky and sensuous scent. It has a grounding, balancing, calming scent and provides numerous health benefits, making it ideal for therapeutic use in cosmetics and aromatherapy. In some such embodiments, the composition of the present technology thus comprises CBD or a derivative thereof, and patchouli oil, or an extract thereof.

In some embodiments, the functional fragrance useful in the compositions of the present technology comprises between about 0.005 wt % and about 10 wt % patchouli oil, or between about 0.025 wt % and about 10 wt % patchouli oil, or between about 0.1 wt % and about 10 wt % patchouli oil, or between about 0.2 wt % and about 10 wt % patchouli oil, or between about 0.1 wt % and about 5 wt % patchouli oil.

In some embodiments, the functional fragrance useful in the compositions of the present technology comprises ho wood oil. Ho Wood Oil is extracted from the leaves of Cinnamomum camphora. It is extremely high in calming linalool and is excellent for promoting a peaceful and relaxing environment. Ho Wood has a woody, floral, and camphorous scent. In some such embodiments, the composition of the present technology thus comprises CBD or a derivative thereof, and Ho wood oil, or an extract thereof.

In some embodiments, the functional fragrance useful in the compositions of the present technology comprises between about 0.005 wt % and about 10 wt % Ho wood oil, or between about 0.025 wt % and about 10 wt % Ho wood oil, or between about 0.25 wt % and about 10 wt % Ho wood oil, or between about 0.1 wt % and about 10 wt % Ho wood oil, or between about 0.1 wt % and about 6 wt % Ho wood oil.

In some embodiments, the functional fragrance useful in the compositions of the present technology comprises grapeseed oil. Grapeseed oil comes from the pressed seeds of grapes. The oil is a by-product of the process of making wine. It is known for its anti-inflammatory, antimicrobial and antioxidant properties. These properties, along with the high amounts of omega chain fatty acids and vitamin E contained in grapeseed oil, have made it a popular topical treatment for skin. In some such embodiments, the composition of the present technology thus comprises CBD or a derivative thereof, and grapeseed oil, or an extract thereof.

In some embodiments, the functional fragrance useful in the compositions of the present technology comprises between about 50 wt % and about 99 wt % grapeseed oil, or between about 75 wt % and about 99 wt % grapeseed oil, or between about 90 wt % and about 98 wt % grapeseed oil, or about 90 wt % grapeseed oil, or about 91 wt % grapeseed oil, or about 92 wt % grapeseed oil, or about 93 wt % grapeseed oil, or about 94 wt % grapeseed oil, or about 95 wt % grapeseed oil, or about 96 wt % grapeseed oil, or about 97 wt % grapeseed oil, or about 98 wt % grapeseed oil, or about 99 wt % grapeseed oil.

In some embodiments, the functional fragrance useful in the compositions of the present technology comprises coconut oil. Coconut oil, or copra oil, is an edible oil extracted from the kernel or meat of mature coconuts harvested from the coconut palm. It has various applications. Coconut Essential Oil is commonly used in aromatherapy for alleviating a number of skin issues, including eczema, psoriasis, dermatitis, and dandruff. It is known to reduce damaging inflammation and moisturize the skin to allow quicker healing. In some such embodiments, the composition of the present technology thus comprises CBD or a derivative thereof, and coconut oil, or an extract thereof.

In some embodiments, the functional fragrance useful in the compositions of the present technology comprises between about 50 wt % and about 90 wt % coconut oil, or between about 50 wt % and about 80 wt % coconut oil, or between about 50 wt % and about 75 wt % coconut oil, or about 50 wt % coconut oil, or about 55 wt % coconut oil, or about 60 wt % coconut oil, or about 65 wt % coconut oil, or about 70 wt % coconut oil, or about 75 wt % coconut oil.

In some embodiments, the functional fragrance useful in the compositions of the present technology comprises marjoram oil. In some embodiments, the functional fragrance useful in the compositions of the present technology comprises between about 0.005 wt % and about 10 wt % marjoram oil, or between about 0.05 wt % and about 10 wt % marjoram oil, or between about 0.1 wt % and about 10 wt % marjoram oil, or between about 0.25 wt % and about 10 wt % marjoram oil, or between about 0.05 wt % and about 5 wt % marjoram oil, or between about 0.1 wt % and about 5 wt % marjoram oil.

In some embodiments, the functional fragrance useful in the compositions of the present technology comprises black pepper oil. In some embodiments, the functional fragrance useful in the compositions of the present technology comprises between about 0.005 wt % and about 10 wt % black pepper oil, or between about 0.05 wt % and about 15 wt % black pepper oil, or between about 0.05 wt % and about 10 wt % black pepper oil, or between about 0.1 wt % and about 15 wt % black pepper oil, or between about 0.1 wt % and about 10 wt % black pepper oil, or between about 0.2 wt % and about 10 wt % black pepper oil.

In some embodiments, the functional fragrance useful in the compositions of the present technology comprises clove oil. In some embodiments, the functional fragrance useful in the compositions of the present technology comprises between about 0.005 wt % and about 10 wt % clove oil, or between about 0.05 wt % and about 10 wt % clove oil, or between about 0.05 wt % and about 5 wt % clove oil, or between about 0.1 wt % and about 10 wt % clove oil, or between about 0.1 wt % and about 5 wt % clove oil.

The compositions of the present technology may include at least one purified cannabinoid and at least one functional fragrance in a weight ratio of purified cannabinoid to functional fragrance that is between about 100:1 and about 1:500, or between about 50:1 and about 1:500, or between about 25:1 and about 1:500, or between about 10:1 and about 1:500, or between about 1:1 and about 1:500, or between about 100:1 and about 1:100, or between about 25:1 and about 1:100, or between about 1:1 and about 1:100, or between about 50:1 and about 1:50, or between about 1:1 and about 1:50, or between about 25:1 and about 1:10, or between about 1:1 and about 1:10.

The compositions of the present technology may include at least one purified cannabinoid and at least one functional fragrance in a weight ratio of purified cannabinoid to functional fragrance that is about 100:1, about 50:1, about 25:1, about 10:1, about 2:1, about 1:1, about 1:2, about 1:3, about 1:4, about 1:5, about 1:6, about 1:7, about 1:8, about 1:9, about 1:10, about 1:20; about 1:25, about 1:35, about 1:50, about 1:75, about 1:100; about 1:110, about 1:125, about 1:200, or about 1:500.

The compositions of the present technology may include at least one purified cannabinoid and at least one functional fragrance in a weight ratio of purified cannabinoid to functional fragrance that is between about 2:1 and about 25:1, or between about 2:1 and about 10:1, or between about 2:1 and about 5:1.

In an embodiment, the compositions include at least one purified terpene and/or terpenoid. Terpenes and terpenoids are the primary constituents of the essential oils or many types of plants and flowers. Terpenes can be converted to a terpenoid, synthetic terpenoid or semisynthetic terpenoid by any known chemical reactions. In some embodiments, the terpenes include, for example, alpha-Pinene, beta-Pinene, beta-Myrcene, alpha-Terpinene, Limonene, beta-Ocimene, Terpinolene, Linalool, Fenchyl Alcohol, Borneol Isomers, Alpha-Terpineol, Trans-caryophyllene, Alpha-humulene, Trans-nerolidol, Guaiol, Alpha-Bisabolol, and combinations thereof. Suitable terpenoids (and substances that include combinations of terpenes and terpenoids) include α-Pinene, β-Pinene, pine, linalool, linalool oxide, linalyl acetate, lavender, black pepper, myrcene, musk, limonene, citrus, terpineol, lilac, geraniol, citronellol, eugenol, citral, farnesol, bakuchiol, nerolidol, wood bark, eucalyptol, mint, borneol, camphor; α-bisabolol, floral; D-3 Carene, pine, camphene, herbal, β-caryophyllene, Borneol, 1,8-cineole, camphene, humulene, limonene, nerolidol, pulegone, terpinolene, α-phellandrene, Δ3-carene, α-terpinene, β-phellandrene, cis-ocimene, terpinolene, β-caryophyllene, α-guaiene, humulene, δ-guaiene, elemene, guaiol, γ-eudesmol, δ-eudesmol, agarospirol, bulnesol, and α-bisabolol. In one embodiment, the at least one purified terpene is selected from linalool, linalool oxide, limonene, geraniol, citronellol, eugenol, citral, farnesol, bakuchiol, derivatives thereof, and combinations thereof.

In an embodiment, the functional fragrance comprises one or more of Linalool, Linalyl Acetate, Vetiverols, Ocimene, Caryophyllene, Terpentine-4-ol, beta-Pinene, d-Limonene, 1-Octen-3-yl acetate, Geraniol, derivatives thereof, and combinations thereof.

In some embodiments, the functional fragrance useful in the compositions of the present technology comprises linalool. Linalool refers to two enantiomers (S-(+)-linalool and R-(−)-linalool) of a naturally occurring terpene alcohol found in many flowers and spice plants. Its characteristic lavender scent with a hint of spiciness is common to over 200 types of plants. These have multiple commercial applications, the majority of which are based on its pleasant scent. Linalool has sedative, anxiolytic, and anticonvulsant properties (Russo, E. B., Br J Pharmacol. 163(7):1344-64, 2011) and may also assist in pain management. Notably, S-(+)-linalool, and R-(−)-linalool have been shown to be as effective as the trazodone hydrochloride in reducing anxiety. Although it is not clear exactly how linalool exerts its effects on the brain, it appears not to act directly on cannabinoid receptors, and may instead act to block excitatory glutamate receptors and/or inhibit acetylcholine signaling Linalool falls under the Generally-recognized-as-safe (GRAS) classification, and has been shown to be safe at doses as high as 120 mg. In some such embodiments, the composition of the present technology thus comprises CBD; and linalool, linalool oxide, or a derivative thereof.

In some embodiments, the functional fragrance useful in the compositions of the present technology comprises limonene. Limonene is a cyclic monoterpene and is the major component in the oil of citrus fruit peels. D-Limonene is a major component of the aromatic scents and resins characteristic for numerous coniferous and broadleaved trees, such as cottonwoods, aspens, spruces, and various pines, firs and cedars. It contributes to the characteristic odor of orange peel, orange juice and other citrus fruits. The D-isomer, occurring more commonly in nature as the fragrance of oranges, is commonly used as a flavoring agent in food manufacturing and as a fragrance in cosmetics and personal care products. D-limonene is the second most widely distributed terpenoid in nature. It has been shown to have anxiolytic and anti-depression activity. It has also been used to make medicines for treating obesity, bronchitis, and cancer, although these effects are not proven. It is also added to medicinal ointments and creams to facilitate skin penetration. It is nontoxic and highly bioavailable. In some such embodiments, the composition of the present technology thus comprises CBD; and limonene or a derivative thereof.

In some embodiments, the functional fragrance useful in the compositions of the present technology comprises geraniol. Geraniol is a monoterpenoid and is the primary component of rose oil, palmarosa oil, and citronella oil. It also occurs in small quantities in geranium, lemon, and many other essential oils. It is used as an agent for flavoring and fragrance in foods and cosmetics. It is also commonly used as an insect repellent. It is closely related structurally to citral and to linalool, which is an isomer. In some such embodiments, the composition of the present technology thus comprises CBD; and geraniol or a derivative thereof.

In some embodiments, the functional fragrance useful in the compositions of the present technology comprises vetiverol. Vetiverol (also known as vetivenol) is a mixture of sesquiterpenoid alcohols obtained from vetiver oil and often used in perfumes. It is inhaled as aromatherapy for nervousness, insomnia, and joint and muscle pain. It is also applied directly to the skin for relieving stress, as well as for emotional traumas and shock, for stings and burns, for arthritis, and to repel insects. In some such embodiments, the composition of the present technology thus comprises CBD; and vetiverol or a derivative thereof.

In some embodiments, the functional fragrance useful in the compositions of the present technology comprises Linalyl Acetate. Linalyl acetate is a naturally-occurring phytochemical found in many flowers and spice plants. It is one of the principal components of the essential oils of bergamot and lavender. Chemically, it is the acetate ester of linalool, and the two often occur in conjunction. It is commonly used in aromatherapy and massage therapy due to its ability to promote relaxation and better mood when inhaled. In some such embodiments, the composition of the present technology thus comprises CBD; and linalyl acetate or a derivative thereof.

In some embodiments, the functional fragrance useful in the compositions of the present technology comprises ocimene. Ocimene is a cannabis terpene carrying a sweet, herbaceous, woody and earthy aroma with hints of citrus. Also found in mint, parsley, basil, mangoes, orchids, oregano and tarragon, ocimene is commonly used in perfumes and fragrances for its sweet and floral aromatic profile. This terpene contributes significantly to the floral odor of various plant species, including the scent of orchid and snapdragon flowers. Ocimene is also a pheromone involved in social regulation in the honey bee colony. It is believed to have many medicinal effects including antifungal, antiviral, and anti-inflammatory effects. In some such embodiments, the composition of the present technology thus comprises CBD; and ocimene or a derivative thereof.

In some embodiments, the functional fragrance useful in the compositions of the present technology comprises Caryophyllene. Caryophyllene is present in many herbs and spices, including black pepper, basil, and oregano, and cannabis. It is a natural bicyclic sesquiterpene that is a constituent of many essential oils, especially clove oil, the oil from the stems and flowers of Syzygium aromaticum, and the essential oil of Cannabis sativa, rosemary, and hops. It is widely considered to provide digestive protection, pain relief, and act as an antibacterial agent. In some such embodiments, the composition of the present technology thus comprises CBD; and caryophyllene or a derivative thereof.

In some embodiments, the functional fragrance useful in the compositions of the present technology comprises Terpentine-4-ol. Terpentine-4-ol is an isomer of terpineol. A primary constituent of tea tree oil, it is obtained as an extract from the leaves, branches, and bark of Melaleuca alternifolia Cheel. In some such embodiments, the composition of the present technology thus comprises CBD; and Terpentine-4-ol or a derivative thereof.

In some embodiments, the functional fragrance useful in the compositions of the present technology comprises beta-Pinene. beta-Pinene has a woody-green pine-like smell and is one of the most abundant compounds released by forest trees. It is useful for relieving pain, inflammation and anxiety. In some such embodiments, the composition of the present technology thus comprises CBD; and beta-Pinene or a derivative thereof.

In some embodiments, the functional fragrance useful in the compositions of the present technology comprises 1-Octen-3-yl acetate. 1-Octen-3-yl acetate is a component of lavender oil. In some such embodiments, the composition of the present technology thus comprises CBD; and 1-Octen-3-yl acetate or a derivative thereof.

In an embodiment, the functional fragrance is an aromatherapy-based fragrance such as, for example, a topical sleep aid, an aerosol spray, and the like. Non-limiting examples of functional fragrances for use in compositions of the present technology include Deep Calm®, Deep Sleep®, Deep Sleep® Pillow Spray, Sleep Plus® Pillow Spray, Love Sleep®, Oriental®, Love to Sleep®, Super Sleep®, Sleep Power®, Muscle Therapy® and combinations thereof (all of the foregoing functional fragrances are commercially available from This Works®, Wimbledon, UK).

In an embodiment, the functional fragrance is in the form of an aerosol spray, such as without limitation a pillow spray. Several pillow sprays are commercially available for use in improving sleep.

The functional fragrance in the compositions of the present technology may include at least one essential oil in an amount from about 1 wt % to about 99 wt % of the total weight of the functional fragrance present in the compositions; or in an amount of between about 10 wt % to about 99 wt %; or in an amount of between about 25 wt % to about 99 wt %; or in an amount of between about 50 wt % to about 99 wt %; or in an amount of between about 75 wt % to about 99 wt %; or in an amount of about 5 wt %, or in an amount of about 10 wt %, or in an amount of about 25 wt %, or in an amount of about 50 wt %, or in an amount of about 75 wt %, or in an amount of about 90 wt %, or in an amount of about 99 wt % of the total weight of the functional fragrance present in the compositions.

The functional fragrance in the compositions of the present technology may include at least one terpene in an amount from about 0.001 mg/mL to about 1 mg/mL. In other embodiments, the composition may comprise at least one terpene in an amount of about 0.001 mg/mL to about 0.95 mg/mL, or about 0.001 mg/mL to about 0.9 mg/mL, or about 0.005 mg/mL to about 0.8 mg/mL. In some embodiments, the composition may comprise at least one terpene in an amount of about 0.01 mg/mL, about 0.15 mg/mL, about 0.02 mg/mL, about 0.25 mg/mL, about 0.03 mg/mL, about 0.35 mg/mL, about 0.04 mg/mL, about 0.45 mg/mL, about 0.05 mg/mL, about 0.55 mg/mL, about 0.06 mg/mL, about 0.65 mg/mL, about 0.07 mg/mL, about 0.75 mg/mL, about 0.08 mg/mL, about 0.085 mg/mL, about 0.09 mg/mL, about 0.95 mg/mL, or about 1 mg/mL. The concentrations listed are the total concentration of all the terpenes in the composition.

In an embodiment, the composition of the present disclosure includes one or more of THC, CBDA, CBD, CBGA, CBG, CBCA, CBC, THCVA, THCV, CBDVA, and CBDV, and one or more of lavender, vetivert, chamomile, patchouli, ho wood, grapeseed, and coconut oil.

In another embodiment, the composition of the present disclosure includes CBD or a derivative thereof, and one or more of lavender, vetivert, chamomile, patchouli, ho wood, grapeseed, and coconut oil.

In another embodiment, the composition of the present disclosure includes purified CBD or a derivative thereof, and one or more of lavender, vetivert, chamomile, patchouli, ho wood, grapeseed, and coconut oil.

In another embodiment, the composition of the present disclosure includes CBD or a derivative thereof, and one or more of lavender, vetivert, chamomile, patchouli, ho wood, grapeseed, coconut oil, marjoram oil, black pepper oil and clove oil.

In another embodiment, the composition of the present disclosure includes purified CBD or a derivative thereof, and one or more of: Linalool, Linalyl Acetate, Vetiverols, Ocimene, Caryophyllene, Terpentine-4-ol, beta-Pinene, d-Limonene, 1-Octen-3-yl acetate, Geraniol, derivatives thereof, and combinations thereof.

In some embodiments, the compositions of the present disclosure include purified CBD or a derivative thereof, a functional fragrance and at least one vitamin. In some implementations of these embodiments, the vitamin is one or more of: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamin), vitamin C (ascorbic acid), vitamin D (calciferols), vitamin E (tocopherols and tocotrienols), and vitamin K (quinones). In some further implementations, the vitamin is Vitamin A (retinol) Vitamins may be present in the compositions of the present disclosure in an amount ranging from between about 0.001 wt % and about 2 wt %, or between about 0.01 wt % and about 1 wt %, or between about 0.05 wt % and about 2 wt %, or between about 0.05 wt % and about 1 wt %.

In some embodiments, the compositions of the present disclosure include purified CBD or a derivative thereof, a functional fragrance and an additional botanical extract. In some implementations of these embodiments, the additional botanical extract may include one or more of angelica extract, avocado extract, tasmannia lanceolata extract, wild yam extract, boswellia spp. extract, fenugreek extract, harpagophytum spp. extract, hydrangea extract, althea extract, arnica spp. extract, aloe extract (also referred to herein as aloe vera extract and aloe vera leaf extract), apricot extract, apricot core extract, ginkgo extract, fennel extract, turmeric extract, oolong tea extract, rose fruit extract, echinacea leaf extract, scutellaria root extract, phellodendron bark extract, goldthread extract, barley extract, hypericum extract, white nettle extract, watercress extract, orange extract, prunus amygdalus dulcis (sweet almond) oil, seaweed extract, carrot extract, artemisia capillaris extract, glycyrrhiza extract, sabdariffa extract, pyracantha fortuneana fruit extract, cinchona extract, cucumber extract, gardenia extract, sasa albo-marginata extract, sophora root extract, walnut extract, grapefruit extract, Simmondsia chinensis (jojoba) seed oil, clematis extract, chlorella extract, mulberry bark extract, gentian extract, black tea extract, yeast extract, burdock extract, fermented rice bran extract, rice germ oil, comfrey extract, cowberry extract, asiasarum root extract, bupleurum falcatum root extract, salvia extract, saponaria extract, bamboo grass extract, crataegus extract, zanthoxylum fruit extract, shiitake mushroom extract, rehmannia root extract, lithospermum root extract, perilla extract, linden extract, filipendula extract, peony root extract, calamus rhizome extract, birch extract, horsetail extract, ivy extract, hawthorn extract, sambucus nigra extract, yarrow extract, peppermint extract, sage extract, mallow extract, cnidium rhizome extract, swertia herb extract, soy extract, jujube extract, wild thyme extract, green tea extract, clove extract, cogon extract, citrus unshiu peel extract, angelica root extract, calendula extract, peach seed extract, bitter orange extract, houttuynia extract, tomato extract, natto extract, ginseng extract, garlic extract, wild rose extract, hibiscus sabdariffa flower extract, ophiopogon tuber extract, parsley extract, witch hazel extract, pellitory extract, isodonis extract, matricaria extract, loquat extract, coltsfoot extract, butterbur scape extract, Poria cocos extract, butcher bloom extract, grape extract, Olea europaea (olive) fruit oil, propolis, luffa extract, safflower extract, peppermint extract, linden extract, peony extract, hop extract, pine extract, Zingiber officinale (ginger) root extract, horse chestnut extract, skunk cabbage extract, sapindaceae extract, balm mint extract, peach extract, cornflower extract, eucalyptus extract, saxifrage extract, coix seed extract, mugwort extract, lavender extract, apple extract, lettuce extract, lemon extract, Chinese milk vetch extract, rose extract and rosemary extract. In one embodiment, the composition comprises one or more of: prunus amygdalus dulcis (sweet almond) oil, Simmondsia chinensis (jojoba) seed oil, Olea europaea (olive) fruit oil, Zingiber officinale (ginger) root extract, and combinations thereof. The additional botanical extract may be present in the compositions of the present disclosure in an amount ranging from between about 0.1 wt % and about 5 wt %, or between about 0.1 wt % and about 4 wt %, or between about 0.1 wt % and about 3 wt %, or between about 0.1 wt % and about 1 wt %.

In some embodiments, the compositions of the present disclosure include magnesium. In some implementations of these embodiments, the magnesium is magnesium chloride. The magnesium may be present in the compositions of the present disclosure in an amount ranging from between about 0.1 wt % and about 5 wt %, or between about 0.1 wt % and about 3 wt %, or between about 0.5 wt % and about 2 wt %, or between about 0.5 wt % and about 1 wt %.

iii) Non-Medicinal Ingredients

In some embodiments, the compositions of the present technology further comprise non-medicinal ingredients. The non-medicinal ingredients should be acceptable/suitable in the sense of being compatible with any other components in the composition and should not be deleterious to the subject. In some instances, the non-medicinal ingredients may be incorporated into the compositions defined herein with the purpose of, for example, improving administration to the subject, improving bioavailability of the components of the composition, adjusting pH of the composition, modifying the texture of the composition, improving the stability of the composition, and the like. Examples of non-medicinal ingredients useful in the compositions of the present technology include, but are not limited to: absorbents, abrasives, anticaking agents, anticlumping agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, perfumes, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, waxes, sunscreens, sunblocks, ultraviolet light absorbers or scattering agents, sunless tanning agents, antioxidants and/or radical scavengers, chelating agents, and sequestrants. Such other materials are known in the art.

The compositions of the present technology may comprise an emollient. For example, the emollient may be selected from one or more of the following classes: Triglycerides (e.g., Medium-chain triglycerides (MCT)), Triglyceride esters which include, but are not limited to, vegetable and animal fats and oils such as castor oil, cocoa butter, safflower oil, cottonseed oil, corn oil, olive oil, cod liver oil, almond oil, avocado oil, palm oil, sesame oil, squalene, kikui oil and soybean oil; Acetoglyceride esters, such as acetylated monoglycerides; Ethoxylated glycerides, such as ethoxylated glyceryl monostearate; Alkyl esters of fatty acids having 10 to 20 carbon atoms which include, but are not limited to, methyl, isopropyl, and butyl esters of fatty acids such as hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate; Alkenyl esters of fatty acids having 10 to 20 carbon atoms such as oleyl myristate, oleyl stearate, and oleyl oleate; Fatty acids having 10 to 20 carbon atoms such as pelargonic, lauric, myristic, palmitic, stearic, isostearic, hydroxystearic, oleic, linoleic, ricinoleic, arachidic, behenic, and erucic acids; Fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; Lanolin and lanolin derivatives such as lanolin, lanolin oil, lanolin wax, lanolin alcohols, lanolin fatty acids, isopropyl lanolate, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, and liquid and semisolid lanolin absorption bases; Polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, sorbitan fatty acid esters, and polyoxyethylene sorbitan fatty acid esters; Wax esters such as beeswax, spermaceti, myristyl myristate, stearyl stearate; Beeswax derivatives such as polyoxyethylene sorbitol beeswax which are reaction products of beeswax with ethoxylated sorbitol of varying ethylene oxide content, forming a mixture of ether esters; Vegetable waxes including, but not limited to, carnauba and candelilla waxes; Phospholipids such as lecithin and derivatives; Sterols including, but not limited to, cholesterol and cholesterol fatty acid esters; and Amides such as fatty acid amides, ethoxylated fatty acid amides, and solid fatty acid alkanolamides.

The compositions of the present technology may comprise a surfactant. The surfactant may be synthesized or isolated from a natural source. For example, the surfactant may be selected from one or more of the following classes: anionic surfactants, nonionic surfactants, cationic surfactants, amphoteric surfactants, plant-derived surfactants, coconut-derived surfactants, palm-derived surfactants, vegetable derived surfactants, coco glucoside, decyl glucoside, hexyl glucoside, capryl glucoside, lauryl glucoside, disodium laureth sulfosuccinate, coco betaine, sodium coco sulfate, Plantapon® SF, soap nuts or berries, aritha, yucca extract, shikakai powder, saponins, and soapwort. In some such embodiments, the compositions of the present technology comprise a saponin. In some such embodiments, the compositions of the present technology comprise a natural food-grade surfactant such as Quillaja saponin (Q-Naturale). In some such embodiments, the compositions of the present technology comprise a synthetic surfactant such as Tween 80.

The compositions of the present technology may comprise a carrier oil. The carrier oil is, optionally, food grade, does not adversely affect product quality (such as appearance, taste, texture, or stability), protects from chemical degradation during storage and distribution, and/or increases bioavailability following application by, for example, aiding skin penetration of a cream. Carrier oils can help stabilize emulsions.

In embodiments of the compositions of the present technology, the carrier oil may be synthesized or isolated from a natural source. In some embodiments, the carrier oil is a natural oil as known in the art, such as an edible vegetable oil. In some alternative embodiments, the carrier oil is a synthetic edible oil, such as a hydrogenated vegetable oil, a medium-chain triglyceride (MCT) oil, and the like. For example, the carrier oil may be selected from one or more of the following classes, without limitation: medium-chain triglycerides (MCT) oil, medium-chain fatty acids (e.g., caproic acid, caprylic acid, capric acid, lauric acid, long-chain triglycerides (LCT oil), long chain fatty acids (e.g., myristic acid, palmitic acid, stearic acid, arachidic acid, linoleic acid), glycerine/glycerol, Maisine® CC, glycerol monolinoleate, coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, arnica oil, peppermint oil, hemp oil, sesame oil, pomegranate seed oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, LabrafacLipophile WL 1349, Labrail M1944, Peceol, Plurol Oliqiue CC 497, Transcutol HP, Tween 80, Gelucire 48/16, and combinations thereof. Carrier oils may also be in the form of an oil powder, such as a plant or animal-derived oil powder (such as rice bran oil powder, coconut oil powder, grape seed oil powder, cranberry seed oil powder, chia seed oil powder, flaxseed oil powder, MCT oil powder, hydrolyzed collagen powder, saw palmetto oil powder, safflower oil powder, evening primrose seed oil powder, fish oil powder, or the like). In some embodiments, the carrier oil is medium-chain triglycerides (MCT), e.g., MCT oil or MCT oil powder.

In some embodiments of compositions of the present technology, the weight ratio of the cannabinoid to carrier oil is about 5:1 or about 1:5. In alternative embodiments, the weight ratio of cannabinoid to carrier oil is about 1:1.

The compositions of the present technology may comprise an emulsifier. The emulsifier may be synthesized or isolated from a natural source. Emulsifiers may be provided in the liquid, granule or powder form. In some embodiments, the emulsifier is provided in the form of an emulsifying powder. A wide range of emulsifiers and emulsifying powders suitable for spray granulation, milling, liquefying, pressing or spray drying may be used. For example, the emulsifying powder may be selected from one or more of the following classes: lecithin, acconon mixtures, capmul MCG, propylene glycol esters, caprol polyglycerol esters, glycerides, glycerol, captex medium chain esters, Kolliphor, castor oil, polysorbate 80, hydroxypropylcellulose (HPC), and the like.

In some embodiments, the compositions of the present technology are formulated for administration by inhalation or for topical administration. For example, the composition may be in the form of a patch, a matrix, a wax, an ointment, a cream, a serum, a balm, a suspension, a lotion, a paste, a gel, a spray, an aerosol, a foam, or an oil. In some such embodiments, the composition includes one or more formulating agents suitable for administration by inhalation or in topical form such as, without limitation, binders, fillers, bulking agents, excipients, pressing agents, press release agents, powders, dispersing agents, disintegrants, lubricants, glidants, surfactants, emulsifiers, solubilizing agents, and fillers.

In some embodiments, compositions described herein are suitable for transdermal administration. In another embodiment, transdermally administrable compositions are adapted for administration in and/or around the abdomen, back, chest, legs, arms, scalp, face or other suitable skin surface and may include formulations of the compositions disclosed herein administered in patches, ointments, creams, suspensions, lotions, pastes, gels, sprays, aerosols, foams, serums, balms, or oils.

In certain embodiments, the compositions of the present technology are suitable for administration by inhalation and/or nasal administration. In an embodiment, compositions are formulated into a spray, e.g., into a form which is easily sprayable. In an embodiment, compositions are dispensed in the form of an aerosol spray.

In certain embodiments, the compositions of the present technology are suitable for administration as a pillow spray. A pillow spray is designed to be sprayed onto a subject's pillow and/or bed linen before the subject goes to sleep; pillow sprays can provide improved sleep by e.g. helping the subject relax, helping the subject fall asleep faster (reducing sleep latency), improving the quality of sleep, providing less restless sleep, improving REM sleep, and/or causing the subject to wake up feeling more refreshed.

In certain embodiments, the compositions of the present technology, or at least a portion thereof, are encapsulated. This delivery mechanism allows the composition to be released consistently over a prolonged time, for example throughout the night, as the encapsulated fragrance remains enclosed until the encapsulate is broken. Breaking the encapsulate allows additional composition or fragrance to be released. Many different encapsulation technologies are available and may be used in the compositions of the present technology. For examples, encapsulates may be synthetic in origin (e.g., polymer shells), may contain animal derivatives (e.g., lecithin), or may contain only plant derivatives (e.g., vegan encapsulates). Encapsulates may be “broken” to release composition or fragrance through heat, moisture, or friction. In one embodiment, the encapsulate is between about 5 to about 25 microns in size. For example, the encapsulate may be about 5 about 10, about 15, about 20, or about 25 microns in size.

In one embodiment, the composition of the present technology, or at least a portion thereof, is encapsulated such that the encapsulate is broken by friction, e.g., by the subject moving about during the night, allowing additional composition or fragrance to be released. In some such embodiments, the encapsulate comprises only plant-derived materials, e.g., is a vegan encapsulate. In one embodiment, the main components of the encapsulate wall include one or more of: Sodium Alginate (CAS 9005-38-3); Calcium Chloride (CAS 10043-52-4, EINECS 233-140-8); Riboflavin 5-Phosphate (CAS 146-17-8, EINECS 205-664-7); Genipin (CAS 6902-77-8); and combinations thereof. In one embodiment, the components of the encapsulate are 100% naturally derived.

In some embodiments, the compositions of the present technology are formulated into a matrix. In certain embodiments, the matrix may be a lipid matrix. Suitable lipid matrices include, without limitation, natural and/or synthetic oils, fatty acids and their derivatives, glycerides, fatty acid esters, glycolized fatty acid esters, fatty alcohols, sterols, waxes, hard fat, and/or combination thereof. Suitable natural oils include, without limitation, vegetable oil such as sunflower oil, olive oil, groundnut oil, and palm oil, as well as hydrogenated vegetable oils, including hydrogenated cottonseed oil. Suitable synthetic oils include, without limit, hydrophobic silicone, cyclomethicones, petroleum waxes or jellies, linear alkanes, lipophilic organic fluorinated oils, perhydrosqualene and/or mixtures thereof. Suitable fatty acids include, without limitation, stearic acid, benzoic acid, citric acid, iumaric acid, lactic acid, and maleic acid. Exemplary glycerides include, without limitation, monoglycerides, diglycerides, triglycerides, and combinations thereof, etc. with saturated or unsaturated chains having carbon numbers from C₆ to C₄₀, e.g. C₁₈ to C₂₄, C₈ to C₃₂, C₁₀ to C₂₄, C₁₀ to C₁₈, C₁₂ to C₁₈, etc.), hemisynthetic glycerides or glyceride derivatives with saturated or unsaturated medium to long chain lengths. Suitable waxes include, without limitation, Carnauba wax, Candelilla wax, Alfa wax, vegetable waxes, rice wax, hydrogenated jojoba wax or floral absolute waxes, beeswaxes and modified beeswaxes, microcrystalline wax, and paraffin wax. Suitable fatty alcohols include fatty alcohols with high molecular weight (e.g. cetanol, myristoyl alcohol, stearyl alcohol). Esters of acids and alcohols with high molecular weight include, without limitation, esters of linear and saturated acids with even carbon numbers from C₁₄ to C₂₀, and linear and saturated alcohols with even carbon numbers from C₁₄ to C₃₂. Suitable lipid matrix materials may also include clays or their oily dispersions, gums of phenylated silicones, starches, and/or fat structuring agents for the purpose of adjusting consistency. The lipid matrix may also include a certain number of compounds such as mineral fillers, to modulate density and plasticity. The mineral fillers may be, for example, talc and/or kaolin. The matrix material may include mixtures of materials, such as mixtures of any of the foregoing.

Compositions of the disclosure containing can be prepared by any technique known to a person of ordinary skill in the art of pharmacy, pharmaceutics, cosmetics, drug delivery, pharmacokinetics, medicine or other related discipline that comprises admixing non-medicinal ingredients with a drug or therapeutic agent, a functional agent, a cosmetic ingredient, and the like.

In some embodiments, compositions of the present technology comprise a penetration enhancing agent for transdermal or topical delivery. Suitable penetration enhancing agents include, without limitation, C₈-C₂₂ fatty acids such as isostearic acid, octanoic acid, and oleic acid; C₈-C₂₂ fatty alcohols such as oleyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

In one embodiment, the compositions described herein comprise an antimicrobial preservative. Suitable antimicrobial preservatives include, without limitation, acids, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The antimicrobial preservative, if present, is present in an amount of from about 0.1% to about 5%, from about 0.2% to about 3%, or from about 0.3% to about 2%, by weight. In some embodiments, the anti-microbial preservative, if present, is present in an amount of about 0.1%, about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

Compositions described herein may also optionally include one or more taste enhancers, such as sweeteners, including aspartame, acesulfame potassium, sucralose and saccharin or taste masking agents, such as flavorings. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate taste enhancer or taste making agent and the relative concentration of the taste enhancer or taste masking agent.

In some embodiments, the non-medicinal ingredients used in the compositions of the present technology are naturally occurring or are naturally derived or are a combination thereof. As used herein, the expression “naturally occurring” refers to a natural product that is delivered in a natural form. The expression “naturally derived”, as used herein, refers to some ingredients derived from nature that have been used to artificially create a product that is delivered in an unnatural form.

iv) Methods of Use

It is known that cannabinoids, including cannabidiol, have anti-insomnia, anxiolytic, antidepressant, anti-inflammatory, calming, and relaxing effects, which may be effective in ameliorating problems commonly associated with insomnia and sleep disorders. Functional fragrances, including aromatherapy treatments and pillow sprays, are known to provide improved sleep and to help users wake up feeling more rested and/or refreshed. As such, in some embodiments of the present technology, the compositions of the present technology are used as agents for the treatment of insomnia and sleep disorders, and for providing improved sleep.

As used herein, the term “improved sleep” refers to improvement in one or more parameters related to sleep quality or quantity, such as, without limitation, total sleep time, sleep efficiency (percent of time spent in bed that a subject is asleep), sleep latency (how fast a subject falls asleep), amount of rapid eye movement (REM) sleep, number of awakenings, sleep latency after awakening, amount of deep sleep, and a subjective feeling of waking up refreshed and/or rested.

In some embodiments of the present technology, there is provided a method of providing improved sleep in a subject, comprising administering to said subject an effective amount of the composition(s) provided herein. There are also provided methods of treating insomnia and other related sleep disorders in a subject, comprising administering to said subject an effective amount of the composition(s) provided herein.

Insomnia, as used herein, may refer to acute insomnia, chronic insomnia, comorbid insomnia, onset insomnia, maintenance insomnia, adjustment insomnia, idiopathic insomnia, non-organic specific insomnia, organic specific insomnia, paradoxical insomnia, psychophysiological insomnia, sleep hygiene insomnia, adjustment insomnia, behavioral insomnia of childhood, idiopathic insomnia, insomnia due to medical condition(s), and insomnia due to mental disorder(s), or a sleep related disorder including, without limit, REM sleep behavior disorder, sleep talking, sleep walking, nightmares, shift work disorder, delayed sleep phase disorder, excessive daytime sleepiness disorder, excessive sleepiness, narcolepsy, excessive sleepiness, restless leg syndrome, periodic limb movements, teeth grinding, and mental health daily.

In some implementations of these embodiments, the improvement of sleep includes treating, reducing, lessening, inhibiting, relieving, alleviating, or improving, one or more of the following in the subject: insomnia or sleeplessness; anxiety; restlessness; nervousness; total time asleep; feeling of waking up rested; feeling of waking up refreshed; sleep quality; reduction of sleep latency (time to fall asleep); sleep efficiency; remaining asleep for longer; amount of REM sleep; latency of REM; reduction in number of awakenings; reduction of sleep latency after awakening; amount of deep sleep; relaxation; daytime wakefulness; less daytime fatigue; better dreams; and combinations thereof.

Suitable dosages may be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Suitable amounts of the cannabidiol (CBD), derivative, intermediate, or prodrug thereof, and combinations thereof used in the dosage forms of the present disclosure will depend upon many factors including, for example, age and weight of an individual, specific cannabidiol (CBD), derivative, intermediate, or prodrug thereof, and combinations thereof to be used, nature of a composition, intended means of delivery, etc. Ultimately, a suitable amount can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations of the compositions disclosed herein is administered in patches, ointments, creams, suspensions, lotions, pastes, gels, sprays, aerosols, foams, or oils.

In one embodiment, compositions described herein are suitable for topical administration. In another embodiment, topical administrable compositions are adapted for administration in and/or around the face, abdomen, back, chest, legs, arms, scalp, or other suitable skin surface and may include formulations of the compositions disclosed herein is administered in patches, ointments, creams, suspensions, lotions, pastes, gels, sprays, foams, or oils.

In one embodiment, compositions described herein are suitable for ocular administration. In another embodiment, compositions described herein that are ocularly administrable may include formulations of the compositions disclosed herein is placed in ointments, suspensions, solutions, gels, or sprays.

In one embodiment, compositions described herein are suitable for nasal administration. In another embodiment, compositions described herein that are nasally administrable may include formulations of the compositions disclosed herein is placed in ointments, suspensions, solutions, lotions, pastes, gels, sprays, or mists.

In one embodiment, compositions described herein are suitable for inhalation administration. In another embodiment, compositions described herein that are inhaled administrable may include formations of the compositions disclosed herein is placed in an inhaler, vaporizer, vape pen, aerosol spray, or the like.

In some implementations of these embodiments, the methods of improving sleep and treating insomnia and/or related disorders provided herein further comprise administration to the subject of one or more additional medicament useful in the treatment of such conditions. In some embodiments, the methods provided herein further comprise administration of, without limitation: Ambien®, Ambien® CR (zolpidem tartrate), Dalmane® (flurazepam hydrochloride), Halcion® (triazolam), Lunesta® (eszopiclone), Prosom® (estazolam), Restoril® (temazepam), Rozerem® (ramelteon), Silenor® (doxepin), Sonata® (zaleplon), Desyrel® (trazodone), Belsomra® (suvorexant), over-the-counter sleeping pills (such as antihistamines, melatonin, herbal formulations, and others, or combinations thereof. Other medicaments useful for treatment of sleep disorders may also be used in combination with the compositions of the present technology. It should be understood that the one or more additional medicament may be administered to the subject prior to, at the same time as (concomitantly), or subsequent to, administration of the composition of the present technology.

As used herein, the term “treating” or “treatment” of any disease, disorder or condition includes, in some embodiments, arresting or reducing the development of the disease, disorder or condition, and/or ameliorating at least one symptom thereof. In certain embodiments “treating” or “treatment” refers to ameliorating at least one physical parameter, which may or may not be discernible by the patient. In certain embodiments, “treating” or “treatment” refers to inhibiting the disease or disorder, either physically, (e.g., stabilization of a discernible symptom), physiologically, (e.g., stabilization of a physical parameter), or both. In certain embodiments, “treating” or “treatment” refers to delaying the onset of the disease or disorder. In certain embodiments, “treating” or “treatment” refers to slowing or halting the progression of the disease or disorder. The term “treating” thus refers to any indicia of success in the treatment or amelioration of an injury, pathology or condition, including any objective or subjective parameter such as for example: abatement; remission; diminishing of symptoms or making the injury, pathology or condition more tolerable to the subject; slowing in the rate of degeneration or decline; making the final point of degeneration less debilitating; improving a subject's physical or mental well-being; slowing or halting disease progression; or, in some situations, healing or curing a disease. The treatment or amelioration of symptoms can be based on objective or subjective parameters, including the results of a physical examination, a patient's report, or any suitable test for the disease or disorder being treated.

In some instances, the compositions of the present technology are used in methods for providing improved sleep and for treating insomnia and related sleep disorders. In some implementations of these embodiments, the methods of the present technology comprise administering the composition of the present disclosure to a subject in need thereof. Symptoms of insomnia that may be used to identify subjects in need thereof and/or to monitor treatment can be analyzed using any appropriate method known in the art capable of measuring changes in identified outcomes as a result of composition of the present disclosure versus placebo and/or composition of the present disclosure versus a conventional sleep drug. By way of example, without limitation, methods can include one or more of a self-reporting study, a diary, a survey, or a biometric test. In particular, methods may include analyzing one or more of the following: measuring sleep/functional outcomes; diary/actigraph: changes in sleep latency, number of times awoken, time awoken, total sleep time, changes in QoL; polysomnography; EEG: changes in sleep stages: i.e., changes in latency to REM sleep, time in REM sleep, time in SWS, time in Stage 1-2 sleep etc.; EOG: changes in sleep stages: Latency and time (REM vs. NREM); EMG: changes periodic limb movements, restless leg syndrome, etc.; ECG: changes in heart rhythm; pulse oximetry: changes in respiratory airflow; imaging; changes in regional cerebral blood flow (SPECT) at night or during the day; ligand neuroimaging studies (PET or SPECT ligand); Multiple Sleep Latency Test (MSLT) (changes in level of daytime sleepiness); Maintenance of Wakefulness Test (MWT) (changes and levels of alertness); Driving Simulator-measure of changes in alertness; Questionnaire/other written assessments; changes in: Epworth Sleepiness Scale; Berlin Questionnaire® (Sleep apnea); Stanford Sleepiness Scale; Sam-Perelli fatigue rating; Insomnia Survey Index (ISI); Morning Eveningness Questionnaire (MEQ); Pittsburgh Sleep Quality Index (PSQI); Toronto Hospital Alertness Test (THAT); Athens Insomnia Scale; Center for Epidemiologic Studies in Depression Scale; Fatigue Severity Scale; Changes in diagnostic qualifications of insomnia disorders and related according to the DSM 5; or measuring secondary outcome changes—some possibly related to sleep: blood sugar, weight, cortisol, working memory, emotion discrimination and expression, reward processing; changes in prescription medication use (i.e. changes in use of ‘Z’ drugs); Dim Light Melatonin Onset (DLMO) Test-measures melatonin in saliva within a specified time (i.e., 8 pm to 3 am); Up- and/or down-regulation of specific receptors (CB₁, CB₂, GABAa, 5-HT1a, adenosine A2A receptors, etc.) or neurotransmitters/enzymes (fatty acid amide hydrolase (FAAH), anandamide, 2-AG, Ach, AchE, 5-HT, GABA, etc.) in test subject or cell culture (including IPSCs); Ketamine or pentobarbital-induced sleep tests with study drug and measure outcomes including: Sleep latency, EEG, EMG, locomotor activity, body temperature, motor coordination, EEG; locomotor activity (possibly including motor coordination); Memory tests (i.e., Morris water maze (MWM), novel object recognition); Emotion/fear regulation (i.e., fear conditioning test); Anxiety/depression tests (i.e., EPM, or tail hang test); EMG; and measure action potentials in brain slices. Further exemplary testing or methods include radiological methods such as magnetic resonance imagery, e.g., fMRI, on the subject. In some embodiments, Insomnia Severity Index (ISI), actigraphy, and sleep diary are used. ISI is a reliable and valid instrument used to quantify perceived insomnia severity (Morin et al., 2011, the disclosure of which is hereby incorporated by reference in its entirety). A score of <8 on the ISI implies no clinical insomnia, 8-14 implies subthreshold insomnia, 15-21 implies clinical insomnia (mild severity), and 22-28 implies severe clinical insomnia Actigraphy is a non-invasive method of monitoring activity/rest cycles by measuring gross motor activity, and can be used as a proxy to measure sleep parameters. Subjects can for example wear a Motionlogger Micro Watch from Ambulatory Monitoring, Inc. (AMI). The Motionlogger devices and AMI's companion Action-W2 software can be used to acquire total time asleep, latency to sleep, longest time awake after sleep onset, total time awake, and sleep efficiency. Sleep diaries can be completed daily, with self-reported scales of -rested after sleep, quality of sleep, sleep latency, and sleep duration. Quality of life in subjects suffering from insomnia can also be assessed by EQ-5D, a clinically-validated questionnaire that produces a standard, single index value as a measurement of health status (van Reenen and Janssen, 2015, the disclosure of which is hereby incorporated by reference in its entirety).

In some embodiments, the compositions of the present technology are used in methods for the treatment of muscle pain. In some implementations of these embodiments, the methods of the present technology comprise administering the composition of the present disclosure to a subject in need thereof. In some further implementations, the compositions of the present technology are used in methods for relieving tired and sore muscles and/or for providing relief from discomfort and muscle tension. In some instances, muscle relief is achieved while nourishing the skin.

In some embodiments, the compositions of the present technology are used in methods for skin care. In some implementations of these embodiments, the methods of the present technology comprise administering the composition of the present disclosure to a subject in need thereof. In some further implementations, the compositions of the present technology are used in methods for moisturizing and hydrating skin. In some instances, the compositions of the present technology are used in methods for achieving one or more of: reducing the appearance of wrinkles, smoothing skin, brightening skin, improving skin cell renewal, and increasing collagen production in skin.

In some instances, the administration comprises topical or transdermal administration, e.g., administration of a form such as a lotion, cream, ointment, gel, paste, liniment or balm, serum, drops, skin patch, spray, aerosol spray, and the like.

In some instances, the administration comprises nasal administration or administration by inhalation, e.g., administration of a form such as a spray, an aerosol, a pillow spray, and the like.

It should be understood that compositions of the present technology can be administered using any device or kit for delivery known in the art. In an embodiment, the compositions described herein are provided in a device for delivery to a subject in need thereof. The device may include any container suitable for holding a maximum amount of the composition, a provisioning mechanism for providing a dose of the composition to the subject, and a metering system for transporting the composition to the provisioning mechanism, such that the amount of composition delivered to the subject is controlled by the metering system. The device may deliver any amount of the composition held in the container. In a further embodiment, the amount delivered to the subject is less than the maximum amount held in the container. In some embodiments, the amount delivered to the subject is the same as the maximum amount held in the container. The delivery of the composition from the device to the subject may be controlled by the subject or by another.

A “subject” may be a human or an animal, such as without limitation a cat, a dog, a monkey, a mouse, a rat, or a rodent. In some embodiments, a subject is a patient suffering from insomnia or another sleep disorder, or otherwise in need of treatment for symptoms or sequelae of insomnia and associated conditions.

In some embodiments, the compositions of the present disclosure are formulated to a form suitable for administration to a subject (e.g., human, animal) In some implementations of these embodiments, the compositions as defined herein are formulated in a dosage form selected from a group consisting of liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, serum, drops, skin patch, vaginal, suppository, pessary, rectal and any combination thereof. In some embodiments the compositions defined herein are formulated in any form suitable for inhalation. In some implementations of these embodiments, the compositions of the present disclosure are formulated in a form suitable for inhalation or nasal administration to a subject, such as a sprayable or aerosol form. In some implementations of these embodiments, the compositions of the present disclosure are formulated in a form suitable for topical or transdermal administration to a subject, such as a cream, lotion, serum or ointment. The most suitable route of administration in any given case will depend on the nature and severity of the condition being treated.

In some embodiments, the compositions of the present disclosure are formulated in a unit dosage form for ease of administration and uniformity of dosage. The term “unit dosage form” refers to a physically discrete unit suitable as unitary dosages for human subjects and other animals, each unit containing a predetermined quantity of active material (e.g., cannabinoid, functional fragrance) calculated to produce the desired effect, in association with excipients and/or other components of the composition. The specification for the unit dosage forms of the technology may vary and are dictated by and directly dependent on (a) the unique characteristics of the active compound(s) and the particular effect to be achieved, and (b) the limitations inherent in the art of formulating such active compound(s) (e.g., cannabinoid).

Suitable dosages of the compositions for use in the methods of the present disclosure will depend upon many factors including, for example, age and weight of an individual, at least one precise event requiring professional consultation, severity of an event, specific composition to be used, nature of a composition, route of administration and combinations thereof. Ultimately, a suitable dosage can be readily determined by one skilled in the art such as, for example, a physician, a veterinarian, a scientist, a psychologist, a cosmetologist, an esthetician, and other professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment or cosmetic outcome or result.

In a particular embodiment, the composition of the present technology comprises from about 3 to about 5 wt % CBD.

In a particular embodiment, the composition of the present technology comprises about 100 mg/mL CBD.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Effect of Fragrance Compositions on Sleep and Wellbeing

Two fragrance compositions were tested for their effect on sleep and wellbeing by evaluating the influence of the fragrances on the brain, using functional magnetic resonance imaging (fMRI) and behavioral studies.

There is a direct connection of olfactory neurons to the limbic and memory centers of the brain, such that the olfactory system may in some ways be viewed as an extension of the limbic system which governs emotions and behaviors (aggression, fear, mating, etc.). It is the only sensory system that connects directly with behavioral centers without requiring process information in other centers such as the thalamus. Hence a direct behavioral response can be elicited to a particular odorant, linked to the memory of that odorant.

Specific olfactory neurons, located in the upper region of the nose, detect inhaled odorous molecules. These molecules attach to specialized receptor proteins triggering an electrical signal traveling to the olfactory bulb. The bulb, located in the front of the brain, connects the nose with the olfactory cortex which processes the olfactory information (reconstructing the dissected scent), prior to projecting to higher sensory centers in the cerebral cortex that control emotions, thoughts and behavior. Different scents may elicit different patterns of activation in the brain, underlying the range of emotional and behavioral responses that smell provokes.

The emotional and behavioral alterations caused by fragrance inhalation may be assessed by monitoring electrical activity of the brain (electroencephalography or EEG), by functional magnetic resonance imaging (fMRI), and by behavioral studies. In particular fMRI provides outstanding spatial resolution and is widely used in many areas of cognitive neuroscience.

Study design. The study was conducted at the Technical University and the Medical Faculty Carl Gustav Carus (Psychotherapy and psychosomatic Medicine) in Dresden (Germany). Thirty participants were tested (15 men and 15 women, aged 20-35 (mean 24.4 SD+/−3.2 years). All of the participants exhibited normal olfactory function regarding threshold, discrimination and identification as controlled with the Sniffing Sticks test (Burghardt, Germany). Functional MRI measurement was performed with a 3 Tesla scanner (Sonata; Siemens, Erlange, Germany). Three fragrances were presented expiration-triggered to both nostrils in a total of 6 runs (2×3 runs). The following two fragrances were tested: “DeepSleep®” (lavender, vetivert, camomile, patchouli, and ho wood) and “Oriental®” (patchouli, ylang & olibanum-frankincense) against a control (“placebo”—Phenylethylalcohol [PEA]). The fragrances were first presented for intensity to a sample of 15 participants and, based on the pretest results, a dilution of 6.25% (diluted in 1,2-propanediol) was chosen for DeepSleep® and Oriental® and a dilution of 100% for the control fragrance in order to achieve semi-intense perception.

Procedure. Six runs of fragrance presentation with about 10 on-off blocks were performed in a randomized order. The exact number of fragrance presentations per run depended on the breathing frequency. Each of the fragrances was hence presented in two runs, in order to enhance statistical power. After each run, the participants were asked to rate the intensity of the odor perception on a scale from 0 to 5 (not intense at all to very intense).

The fragrances were applied used a computer-controlled olfactometer. Stimuli were embedded in a constant flow of odorless air (total flow 2 l/min). The stimuli were directed through a small tube from the olfactometer to the participants' noses. Hundred fifty volumes per run were acquired by means of a 38 axial-slice matrix (TR:3000 ms/TE: 40 ms, FA 90; matrix=348*348). Following the fMRI sessions, a T1-weighted image was acquired in sagittal direction by using a T1-MPR sequence (TR: 1890 ms/TE: 3.24 ms; matrix 238*348). This scan was run to allow individualized brain normalization in the later statistical analysis.

Statistical analysis. Intensity ratings were analyzed using SPSS 22. The main effect of odor quality and repetition as well as the interaction effect were modulated in repeated measurements ANOVA. Post hoc tests were computed as t-tests for depended measurements and are presented Bonferroni-corrected. Neural data analysis was performed with the SPM 12 software (Statistical Parametric Mapping; Wellcome Department of Imaging Neuroscience, Institute of Neurology at University College London (UCL], UK)., implemented in Matlab R2015b (Math Works Inc, Natick, Ma, USA), following spatial pre-processing with the same software (spatial filtering: high pass filter 128 Hz, registering, realignment, co-registration between functional and structural images, normalization using segmentation procedure, smoothing by means of 6×6×6 mm3 FWHM Gaussian Kernel).

SPM matrices reflecting the ON-OFF differences were calculated for each session, based on the general liner modeling approach. As the fragrances proved not to be perceived as semi-intense in the scanner, all activations were corrected for the individual intensity rating. Analysis was based on t-test with global height threshold p<0.001 for the overall olfactory activation. All activation coordinates are presented in MNI space (Montreal Neurological Institute [Defined a standard brain by using a large series of MRI scans on normal brains]).

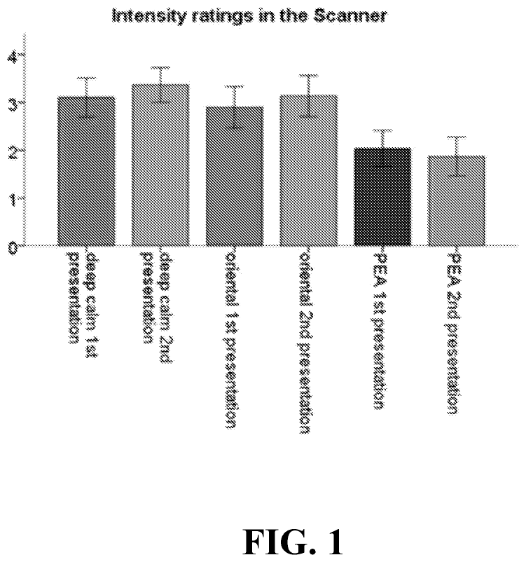

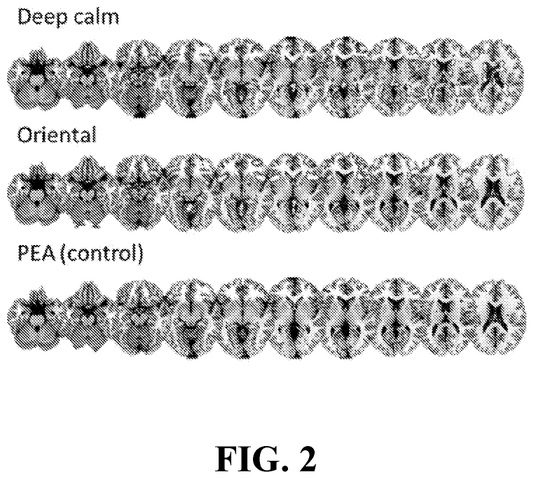

General Results. (1) Perception of the fragrances within the scanner. There was a significant effect of odor quality on intensity ratings (F[2,29]=29,3, p<0.001), but no effect of repeated presentation (F[1,30]=1,6, p=0.21) and no significant interaction effect ([2,29]=1,6, p=0.20, FIG. 1 (note DeepSleep® is referred to as “Deep Calm” in the figure)). Post hoc test revealed that both of the fragrances, DeepSleep® and Oriental®, were rated as significantly more intense than the control fragrance of PEA. (2) Overall neural activation. The presentation of the fragrances was related to a clear activation of olfactory relevant areas, such as the orbitofrontal cortex, amygdala and insula (FIG. 2). The middle cingulate cortex was responsive as well, suggesting that the fragrances evoked hedonic responses. Furthermore, there were neural response patterns in the hLP1 region which suggests a trigeminal perception in all of the odors.

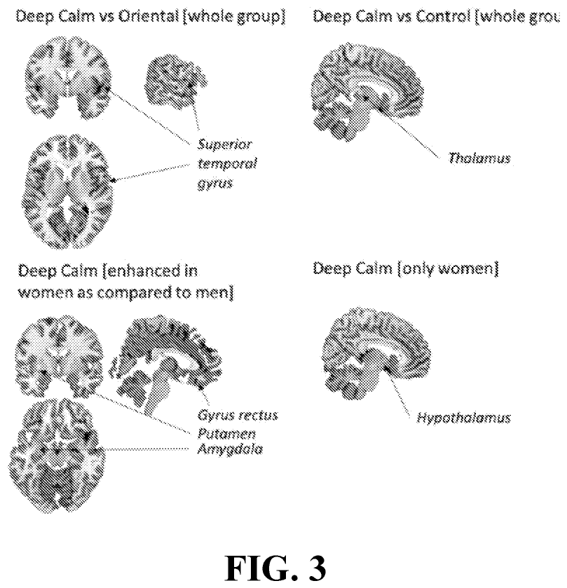

Results for DeepSleep®. (1) DeepSleep® vs. Control. Corrected for differences in intensity ratings, DeepSleep® was related to higher Blood Oxygen Level Dependent (BOLD) signal change in the thalamus and middle frontal gyms. None of the primary or secondary olfactory projection areas and none of the trigeminal projection areas proved to be significantly enhanced in DeepSleep® as compared to the control odor. (2) DeepSleep® vs. Oriental®. Corrected for differences in intensity ratings, DeepSleep® was related to higher BOLD signal change in the right superior temporal gyms, an area which is related to social cognition. (3) Hypothalamic activation. It was specifically checked whether the fragrance was related to enhanced hypothalamic activation. For this purpose a Region of Interest (ROI) sphere of 6 mm (around 3 0-8; MNI space) was created and a liberal threshold of p<0.01 was applied. For DeepSleep®, this region proved to be activated with very low effect size in women [T=2.5, p=0.007, k=1], but not in men. Furthermore, the activation was significantly higher in women as compared to men [T=2.7, p=0.004, k=4]. (4) Sex-differentiated effects. Men showed no significantly enhanced activations as compared to women. Women however showed a more pronounced activation of the left putamen (cl1), the rectal gyms (cl2), the superior frontal gyms and the left and right amygdala (cl4). Results are shown in FIG. 3.

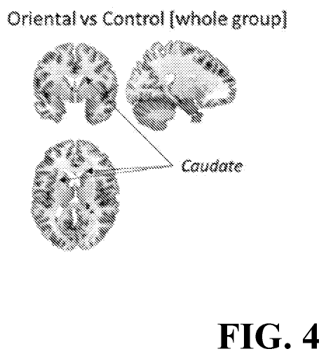

Results for Oriental®. (1) Oriental® vs. Control. Corrected for differences in intensity ratings, Oriental® was related to enhanced BOLD signal change in the right caudate and left caudate and a cluster which bordered the right thalamus. None of the primary or secondary olfactory projection areas and none of the trigeminal projection areas proved to be significantly enhanced in Oriental® as compared to the control odor. (2) Oriental® vs DeepSleep®. There was no enhanced activation in Oriental® odor as compared to the DeepSleep® odor. (3) Hypothalamic activation. It was specifically checked, whether the fragrance was related to enhanced hypothalamic activation. For this purpose a ROI sphere of 6 mm (around 3 0-8; Montreal Neurological Institute (MNI) space (The MNI defined a standard brain by using a large series of MRI scans on normal brains) was created and a liberal threshold of p<0.01 was applied. For Oriental®, there was a slight activation in men [T=2.6, p=0.005, k=2], but not in women. However, the gender differences were not significant. Results are shown in FIG. 4.

Conclusion. All tested fragrances showed the expected activations in olfactory eloquent areas. The study showed a significant difference between DeepSleep®, Oriental® and PEA. DeepSleep® was the most interesting odor, as it related to (a) the highest neural central activation in (b) very interesting areas of the brain. Oriental® in turn was more effective than PEA, impacting more brain areas than PEA. Activation of the anterior intra-parietal sulcus (an area involved in somatosensory integration) suggests that the odor stimulated the trigeminal nerve, which fits with the odor being perceived as slightly stinging. A higher dilution of the odors would reduce this trigeminal activation. A good balance is needed in order to get a well perceived odor without trigeminal activation.

No effect of repeated exposure to the fragrance was observed. Each odor was presented twice and presentations were interrupted by presentation of a different odor (e.g., DeepSleep-Oriental-PEA followed by a block DeepSleep-PEA-Oriental, etc.). There were no significant differences in neural activation between both presentations of the same odor. This phenomenon of low habituation is known in olfaction. However, olfaction is also known for rapid adaptation. This means that an odor which is presented uninterrupted for a long time will not be perceived anymore. We therefore chose a block design with rather short blocks.

Activation of the middle cingulate cortex, especially by DeepSleep®, suggested that the fragrances evoked hedonic responses, being well aware that there are multiple areas involved in hedonic coding. Activation of the caudate (Oriental®) and the putamen (DeepSleep®) areas points to reward processes (both structures form the basal ganglia containing dopaminergic neurons; among its many functions (motor processes, sleep, emotion), the putamen-caudate forms one of the major brain structures composing the reward system). Men showed a significantly higher activation in the left inferior frontal gyrus, right inferior parietal lobule, right marginal gyms and left temporal pole as compared to women. Women on the other hand showed enhanced activation as compared to men in the right hippocampus, which may indicate an effect on prosocial behavior and an impact on sleep. Finally, DeepSleep® activation patterns in the superior temporal gyms suggest an involvement of social cognition related brain areas, which may indicate a more “receptive” attitude to social stimulation.

In summary, a pillow spray was tested containing both free and encapsulated formats of the functional fragrance ‘Deep Sleep’, a blend of Lavender, Chamomile, Vetivert and Patchouli essential oils in a fMRI study. The study was completed on 30 people compared to a placebo (synthetic lavender) fragrance. The ‘Deep Sleep’ blend was shown to positively affect specific regions of the brain (Thalamus, Hippocampus, the middle cingulated cortex) intimately associated with emotions, pleasure and calmness, with the potential to bring the mind into a pre-disposition towards sleep. Taken together, the results suggest that the tested fragrances, and particularly DeepSleep®, may evoke a feeling of social comfort, trust and calmness, and are consistent with positive effects on mood, sleep, and/or prosocial behavior.

### Example 2—Clinical Study of a Fragrance Using Polysomnography and Actigraphy

A clinical study using Polysomnography (PSY) and Actigraphy was conducted to assess the fragrance level, delivery mechanism and the impact on sleep quality and REM sleep for a commercially-available pillow spray. Three pillow sprays with different levels of fragrance were tested: 1: free fragrance only; 2: encapsulated fragrance only; and 3: Sleep Plus® Pillow Spray (This Works®, Wimbledon, UK) with both free and encapsulated fragrance. These were compared to use of no spray. The PSY was completed on 4 subjects and the actigraphy was completed on 7 subjects. The PSY results indicated Pillow Sprays 2 (encapsulated fragrance only) and 3 (Sleep Plus® Pillow Spray, mix of free and encapsulated fragrance) had significant results and suggest improvement in the variables considered (total sleep time, sleep efficiency, sleep latency, latency to REM, number of awakenings, and amount of deep sleep and REM).

The following describes the study in detail: The effects of three different formulations or combinations of a functional fragrance (a pillow spray referred to herein as Deep Calm®) were studied in a single-blind (participants unaware of the spray content), pseudo-randomized (different ranking presentation) clinical study using Polysomnography (PSY) and Actigraphy. The following three formulations were tested: Odor 1: 1% Deep Calm®; Odor 2: 3.24% Vegan encapsulated Deep Calm®; Odor 3: 3% Deep Calm®+3.24% Vegan encapsulated Deep Calm®.

Subjects. Seven subjects, 4 women and 3 men, participated in the actigraphy (AW) study, while 2 women and 2 men were recorded with polysomnography (PSG). All the subjects (>19 year old) were healthy and did not complain of sleep problems. The participants were instructed on how to use the fragrances: After shaking the bottle well they should spray 3 times at arm length onto pillow and twice at arm length spray the top of the duvet and around the areas that will be nearest to their face before going to bed.

Study design. The trial design included both subjective (self-reported) and objective (physiological) measurements. Two types of recordings were used on two different groups of participants: one group was, during a 2-week period, continuously recorded with actigraphy (AW) (to study sleep quality), while another group was recorded 4 times with comprehensive polysomnography (PSG) at home (to study sleep structure and stages).

Methods. (1) Subjective measures. During the entire test period, the participants in the actigraphy group filled in a sleep/day diary reporting sleep perception together with any comments about their night's sleep and any environmental changes of importance. Each morning, they also reported on a “Morning questionnaire” their ‘sleep quality’ on a visual analog scale (VAS; with the end-anchors ‘Very good’ and ‘Very bad’), as well as on the Karolinska Sleepiness Scale (KSS; 1=very alert to 9=very sleepy). At the end of the study the participants filled in an “Evaluation questionnaire” reporting their subjective feelings about using the aromas on a VAS scale, with specific questions about their sleep quality and occurrence of dreams and type of dreams. The subjects of the PSG group used the same morning questionnaire after each PSG recording and the evaluation questionnaire at the end of the test, but they did not use a sleep diary.

(2) Objective measures. (A) Actigraphy: The actigraph “watch” consisted of an accelerometer (Actiwatch; Cambridge Neurotechnology Ltd, Cambridge, UK) that was worn on the same wrist continuously during the whole 2 week test period. Data were stored in the watch unit. Analyses of patterns and frequencies of movements were done by validated algorithms for the recognition of basic sleep-wake patterns. The participants' time-to-bed and waking-up time were reported in their sleep diaries, which defined their sleep periods. The main analyses were conducted on these sleep periods. The first day the participants received the instructions, the actigraph as well as the various questionnaires and the 3 aromas to be tested, marked “1”, “2” and “3”. The ranking on which aroma to use as first, second and third was pseudo-randomized. The subjects were instructed to maintain their ordinary lifestyle and activities however avoiding major irregularities in their sleep-wake pattern such as going to bed late and getting up late during the weekend. During the whole study participants filled in every morning a “Morning Questionnaire” reporting sleep quality and marked their sleep diary, according to instructions as follows: The first 2 nights they slept as usual, using a pillowcase which should not smell anything; The following 3 nights (nights 3-5, “active period 1”), each night at bedtime, they sprayed the selected aroma on the pillowcase according to instructions; Nights 6 and 7 (“washing period”), changing the pillowcase (not smelling) they slept as usual without any spray; The next 3 nights (night 8-9-10, “active period 2”) they repeated the same procedure as nights 3-5 using another aroma; Nights 11 and 12 (“washing period”) subjects repeated the previous washing period, sleeping on a new pillow case without spray; Finally the last 3 nights (“active period 3”) they repeated again the same procedure as in “active period 1” spraying the last aroma on the pillow case; The last day they filled a new questionnaire “Final Evaluation” reporting their experience and evaluating the different aromas.

(B) PSG: PSG was measured using a professional multi-channel ambulatory polygraph (Noxturnal, Resmed) that recorded electroencephalography (electrical brain activity), electromyography (muscle activity), electrooculography (eye movements), electrocardiography (heart beat) as well as body movements and respiratory activity. Each participant was recorded at night at home. After waking up in the morning the subjects filled in the morning questionnaire Four nights were recorded: The first night, subjects were recorded while sleeping as usual, in order to provide a baseline and address the “first night effect”. The 3 next PSGs were done while the subjects used the selected aromas, sprayed at bedtime on a fresh pillow case as done for the actigraphy, and completing the questionnaire in the morning. Following the 4 PSGs the subjects filled in the evaluation questionnaire. The following variables were considered: wake after sleep onset (WASO; in mins), total sleep time (TST), sleep efficiency, sleep latency, latency to rapid eye movements (REM), number of awakenings, amount of deep sleep and REM, arousal index.

Actigraphy Results. Actigraphy was used to determine only sleep variables; therefore only the sleep period was analyzed, with the time-in-bed adjusted according to each participant's sleep diary. Seven subjects received the actigraph, however only data from 6 were analyzed, as one subject had corrupted data. Of the many parameters analyzed (many redundant) the following were retained: sleep time (percent), sleep efficiency, wake bouts, immobile time (%) and fragmentation index, all reflecting the quality of sleep. Results are given in Table 1. The best results on sleep quality were obtained with formulations 2 and 3 (Odors 2 and 3). In some cases, paradoxical increased activity was observed.

TABLE 1
Actigraphy results.

| | No odor | Odor 1 | Odor 2 | Odor 3 |
|---|---|---|---|---|
| Subject 1 (Male) | | | | |
| Sleep % | 91.77 | 87.33 | 90.33 | 89.33 |
| Sleep efficiency | 87.32 | 83.67 | 86 | 80.67 |
| Wake bouts | 22.44 | 27.67 | 22.33 | 26 |
| Immobile time % | 93.43 | 91 | 94 | 91 |
| Fragmentation | 24.27 | 33 | 15.67 | 31.67 |
| index | | | | |
| Subject 2 (Male) | | | | |
| Sleep % | 80.73 | 71.93 | 67.77 | 75.57 |
| Sleep efficiency | 64.47 | 56.80 | 53.50 | 59.67 |
| Wake bouts | 38.33 | 37 | 35.67 | 41.67 |
| Immobile time % | 71.25 | 62.27 | 65.13 | 70.97 |
| Fragmentation | 78.03 | 83.10 | 80.50 | 75.90 |
| index | | | | |
| Subject 3 (Female) | | | | |
| Sleep % | 91.15 | 89.63 | 87.67 | 92.20 |
| Sleep efficiency | 80.08 | 81.17 | 80.57 | 86.13 |
| Wake bouts | 33.8 | 31 | 39 | 35 |
| Immobile time % | 91.6 | 91.2 | 88.4 | 91.5 |
| Fragmentation | 32.5 | 28.1 | 41.2 | 31.5 |
| index | | | | |
| Subject 4 (Female) | | | | |
| Sleep % | 84.67 | 77.77 | 87.20 | 81.57 |
| Sleep efficiency | 75.37 | 72.73 | 74.53 | 73.50 |
| Wake bouts | 58.83 | 60.33 | 38 | 58.67 |
| Immobile time % | 83.15 | 81.53 | 87.87 | 83.50 |
| Fragmentation | 54.35 | 53.97 | 45.30 | 47.80 |
| index | | | | |
| Subject 5 (Female) | | | | |
| Sleep % | 93.58 | 93.77 | 90.43 | 96.43 |
| Sleep efficiency | 83.58 | 85.9 | 84.43 | 88.17 |
| Wake bouts | 24 | 21 | 24 | 21 |
| Immobile time % | 93 | 93 | 90 | 96 |
| Fragmentation | 30 | 27 | 31 | 17 |
| index | | | | |
| Subject 7 (Male) | | | | |
| Sleep % | 85 | 90.5 | 88.5 | 90.3 |
| Sleep efficiency | 82.5 | 89 | 86.5 | 90 |
| Wake bouts | 44.5 | 31 | 37.5 | 30.5 |
| Immobile time % | 87 | 92.5 | 92 | 91.3 |
| Fragmentation | 48.5 | 20 | 29.5 | 29.5 |
| index | | | | |

PSG Results. PSG results are shown in Table 2. The selected variables represent sleep quality and efficiency with analysis of the occurrence and duration of various sleep stages as well as awakenings. WASO: wake after sleep onset), TRT: total recording time. The best PSG results were also obtained with formulations 2 and 3 (Odors 2 and 3).

TABLE 2
PSG results.

| | No odor | Odor 1 | Odor 2 | Odor 3 |
|---|---|---|---|---|
| Subject 1 (Male) | | | | |
| Total sleep time | 367 | 404 | 436 | 381 |
| (TST min) | | | | |
| Sleep efficiency | 90.6 | 87.8 | 90.8 | 79.4 |
| REM latency (min) | 56 | 51 | 33 | 105 |
| WASO (min) | 33.6 | 52.4 | 24.5 | 18.5 |
| Deep sleep | 18.1 | 27 | 26.9 | 27.7 |
| duration (% TST) | | | | |
| REM sleep | 17.1 | 24.8 | 24.9 | 16.5 |
| duration (% TST) | | | | |
| Wake (% TRT) | 9.3 | 11.4 | 5.10 | 5.20 |
| Number of REM | 5 | 5 | 4 | 4 |
| cycles | | | | |
| Subject 2 (Male) | | | | |
| Total sleep time | 464 | 422 | 455 | 521 |
| (TST min) | | | | |
| Sleep efficiency | 96.6 | 87.8 | 94.7 | 86.9 |
| REM latency (min) | 91 | 106 | 78 | 72 |
| WASO (min) | 9 | 18 | 8 | 8.6 |
| Deep sleep | 25.2 | 21.6 | 27.1 | 22.5 |
| duration (% TST) | | | | |
| REM sleep | 24.1 | 20.2 | 24.4 | 22.8 |
| duration (% TST) | | | | |
| Wake (% TRT) | 3.3 | 4.6 | 3.3 | 7.2 |
| Number of REM | 5 | 3 | 5 | 5 |
| cycles | | | | |
| Subject 3 (Female) | | | | |
| Total sleep time | 325 | 495 | 499 | 405 |
| (TST min) | | | | |
| Sleep efficiency | 74.6 | 82.4 | 91.2 | 88 |
| REM latency (min) | 107 | 98 | 86 | 34 |
| WASO (min) | 39.5 | 26.5 | 7 | 11 |
| Deep sleep | 88.5 | 82 | 109 | 62.5 |
| duration (% TST) | | | | |
| REM sleep | 11.6 | 24.8 | 20.7 | 17.2 |
| duration (% TST) | | | | |
| Wake (% TRT) | 10.8 | 6.8 | 2 | 2.4 |
| Number of REM | 2 | 5 | 4 | 3 |
| cycles | | | | |
| Subject 4 (Female) | | | | |
| Total sleep time | n.d.¹ | 311 | 395 | 350 |
| (TST min) | | | | |
| Sleep efficiency | n.d.¹ | 70 | 59.8 | 80.3 |
| REM latency (min) | n.d.¹ | 67 | 147 | 78 |
| WASO (min) | n.d.¹ | 96.6 | 96.5 | 81 |
| Deep sleep | n.d.¹ | 16.4 | 19.7 | 24.5 |
| duration (% TST) | | | | |
| REM sleep | n.d.¹ | 19 | 27.8 | 29.3 |
| duration (% TST) | | | | |
| Wake (% TRT) | n.d.¹ | 23.8 | 14.2 | 18.8 |
| Number of REM | n.d.¹ | 4 | 5 | 4 |
| cycles | | | | |

¹The first recording of Subject 4 was unreliable.

Subjective evaluation results. Table 3 shows data from 7 actigraphy subjects and 3 PSG subjects (the report from subject 3 was unreliable) on the subjective impact of the tested odors.

The lower the value, the better compared to the habitual condition, with number 5 meaning no difference, and less than five meaning a more positive feeling or experience. The results indicate a preference for odors 2 and 3, with odor 3 dominant (AW: actigraphy subject; PSG: PSG subject). Subjects indicated that the tested odors were relaxing and noted improvements in sleep quality, particularly deeper sleep, waking up less often, and falling asleep again faster.

TABLE 3
Subject evaluation results.

| | AW | AW | AW | AW | AW | AW | AW | PSG1 | PSG2 | PSG4 |
|---|---|---|---|---|---|---|---|---|---|---|
| Subject | Male | Male | Female | Female | Female | Female | Male | Female | Male | Female |
| Sleep | 2 | 1 | 3 | 1 | 1 | 1 | 1 | 3 | 1 | 1 |
| Comfort | | | | | | | | | | |
| Sleep quality | 2 | 5 | 4 | 1 | 1 | 1 | 5 | 4 | 1 | 1 |
| Sleep latency | 2 | 1 | 5 | 2 | 2 | 1 | 1 | 5 | 2 | 2 |
| Inconvenience | 2 | 1 | 1 | 1 | 1 | 1 | 1 | 1 | 1 | 1 |
| Sleep | 2 | 4 | 4 | 1 | 2 | 3 | 4 | 4 | 1 | 2 |
| maintenance | | | | | | | | | | |
| Easier to relax | 2 | 2 | 3 | 1 | 1 | 3 | 2 | 3 | 1 | 1 |
| Secure feeling | 2 | 4 | 3 | 1 | 1 | 1 | 4 | 3 | 1 | 1 |
| Morning | 2 | 4 | 8 | 2 | 3 | 4 | 4 | 8 | 2 | 3 |
| awakening | | | | | | | | | | |
| Dream | 5 | 5 | 3 | 1 | 5 | 1 | 5 | 3 | 1 | 5 |
| amount | | | | | | | | | | |
| Vivid dreams | 5 | 5 | 1 | 1 | 5 | 1 | 5 | 1 | 1 | 5 |
| Dream quality | 5 | 5 | 8 | 4 | 5 | 2 | 5 | 8 | 4 | 5 |
| Energy | 2 | 5 | 8 | 3 | 3 | 4 | 5 | 8 | 3 | 3 |
| Discomfort | 0 | 1 | 1 | 3 | 1 | 1 | 1 | 1 | 3 | 1 |
| after | | | | | | | | | | |
| awakening | | | | | | | | | | |
| Impact | 3 | No | No | No | No | 2 | No | No | No | No |
| decrease | | | | | | | | | | |
| Aroma | 2 | 3 | 3 | 3 | 3 | 3 | 3 | 3 or 1 | 3 | 3 |
| preference | | | | | | | | | | |

Conclusions. The study results indicate the tested odors had a positive impact on sleep, both objectively and subjectively. Odors 2 and 3 seemed to have the highest impact both on subjective and objective measures, with Odor 3 seemingly having the highest effect. Odor 3 was also reported by many participants to trigger vivid and unusual dreams. The paradoxical increased activity observed with some subjects in the actigraphy group as well as the sleep fragmentation may be associated to the vivid dreams reported especially with Odor 3. It is noted that antidepressants are known to affect dream recall and content by having an impact on REM (disruption or “rebound” effect after a decrease of REM sleep). In this study, with a very small sample of participants, some impact on REM sleep (latency and duration) could be observed with odors 2 and 3.

Studies can also be conducted to test Sleep Plus® Pillow Spray against a placebo (Synthetic Lavender) to assess the following variables: wake after sleep onset (WASO; in mins), total sleep time (TST), sleep efficiency, sleep latency, latency to rapid eye movements (REM), number of awakenings, amount of deep sleep and REM, and arousal index. Studies can be double blind. Results can also be measured by Self-Perception Questionnaires that will be completed before and after use of the test composition. A trial will be conducted on 100 healthy subjects aged 18 or over who experience disjointed sleep patterns and who have not used any sleep-related prescription medication for 6-12 months before the study.

### Example 3—Sleep-Improving Compositions

Examples of compositions of the present technology are as follows.

Bath Oil Composition

| Ingredient | Wt % |
|---|---|
| Coconut essential oil | 50-75 |
| Lavender essential oil | 1-10 |
| Ho Wood essential oil | 0.1-10 |
| Patchouli Essential oil | 0.1-5 |
| Vetiver essential oil | 3.5-10 |
| Chamomile essential oil | 1.9-5 |
| CBD isolate | 1-5 |
| Tocopherol | 0.01-1 |

Heated the coconut oil to 60-70° C. Added CBD and mixed until fully melted and incorporated into the product. Cooled to 35-45° C. At 45° C. added fragrance and tocopherol, mixing to ensure fully incorporated.

Night Oil composition

| Ingredient | Wt % |
|---|---|
| Grapeseed essential oil | 90-98 |
| Lavender essential oil | 0.3-5 |
| Ho Wood essential oil | 0.25-10 |
| Patchouli Essential oil | 0.1-5 |
| Vetiver essential oil | 0.1-10 |
| Chamomile essential oil | 0.1-5 |
| CBD isolate | 1-5 |
| Tocopherol | 0.01-1 |

Heated grapeseed oil to 60-70° C. Added CBD and mixed until fully melted and incorporated into the product. Cooled to 35-45° C. At 45° C. added fragrance and tocopherol, mixing to ensure fully incorporated.

### Example 4—Muscle Therapy Compositions

An example of a composition of the present technology for improving muscle relaxation and muscle pain management is as follows.

Muscle Therapy Composition

| Ingredient | Wt % |
|---|---|
| Marjoram essential oil | 0.3-5 |
| Black Pepper essential oil | 0.25-10 |
| Clove Essential oil | 0.1-5 |
| CBD isolate | 1-5 |
| Magnesium | 0.5-2 |
| Ginger root extract | 0.1-5 |
| Non-medicinal ingredients | q.s. |

### Example 5—Body Care Compositions

An example of a composition of the present technology for body care is as follows.

Body Care Composition

| Ingredient | Wt % |
|---|---|
| Lavender essential oil | 0.025-5 |
| Ho Wood essential oil | 0.025-10 |
| Patchouli Essential oil | 0.025-10 |
| Vetiver essential oil | 0.025-10 |
| Chamomile essential oil | 0.025-5 |
| CBD isolate | 1-5 |
| Retinol | 0.05-1 |
| Non-medicinal ingredients | q.s. |

### REFERENCES

Morin C M, Belleville G, Belanger L, Ivers H. 2011. The Insomnia Severity Index: Psychometric indicators to detect insomnia cases and evaluate treatment response. Sleep: 34 (5): 601-8.
van Reenen M, Janssen B. 2015. Version 2.1: EQ-5D-5L User Guide: Basic information on how to use the 5Q-5D-5L instrument. EuroQol Research Foundation. The Netherlands.

All cited references are herein expressly incorporated by reference in their entirety to the extent they are consistent herewith.

Whereas particular embodiments have been described above for purposes of illustration, it will be appreciated by those skilled in the art that numerous variations of the details may be made without departing from the disclosure as described herein.

1. A composition comprising:
at least one purified cannabinoid or a derivative thereof; and
at least one functional fragrance.

2. The composition of claim 1, wherein the at least one purified cannabinoid is: cannabigerol ((E)-CBG C-5), cannabigerol monomethyl ether ((E)-CBGM C-5A), Cannabigerolsäure A ((Z)-CBGA C-5A), Cannabigerovarin (((e)-CBGV C-3), Cannabigerolsäure A (e)-CBGA C-5A), A Cannabigerolsäure monomethyl ether ((e)-CBGAM C-5A), Cannabigerovarinsäure A ((e)-CBGVA-C3A); cannabichromene (CBC-C5), Cannabichromensäure A (CBCA C-5A), Cannabichromevarin (CBCVC-3), Cannabichromevarinsäure A (CBCVA-C3A); cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3); Cannabinodiol-like (CBND): Cannabinodiol (CBND C-5), Cannabinodivarin (CBND C-3); Tetrahydrocannabinol-like (THC): Δ⁹-tetrahydrocannabinol (Δ⁹-THC-C5), Δ⁹-tetrahydrocannabinol-C4 (Δ⁹-THC-C4), Δ⁹-tetrahydrocannabivarin (Δ⁹-THCV-C3), Δ⁹-Tetrahydrocannabiorcol (Δ⁹-THCO-C-1), Δ⁹-Tetrahydrocannabinolsäure (Δ⁹THCA-C-5A), Δ⁹-Tetrahydrocannabinolsäure B (Δ⁹THCA-C-5B), Δ⁹-Tetrahydrocannabinolsäure-C4 (Δ⁹THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1 A and/or B), (−)-Δ⁸-trans-(6aR,10aR)-Δ⁸-tetrahydrocannabinol (Δ⁸-THC-C5), (−)-Δ⁸-trans-(6aR,10aR)-Tetrahydrocannabinolsäure A (Δ⁸-THCA-C5 A); (−)-(6aS,10aR)-Δ⁹-tetrahydrocannabinol ((−)-cis-Δ⁹-THC-C5); Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5) Cannabitriol-type (CBT): (−)-(9R,10R)-trans-Cannabitriol ((−)-trans-CBT-C5), (+)-(9S,10S)-Cannabitriol ((+)-trans-CBT C-5), (±)-(9R,10S/9S, 10R)-Cannabitriol ((±)-cis-CBT-C5), (−)-(9R,10R)-trans [10-O-ethyl-cannabitriol] ((−)-trans-CBT-OEt-C5), (±)-(9R,10R/9S,10S)-Cannabitriol-C3 ((±)-trans-CBT-C3), 8,9-dihydroxy-Δ6a (10a) tetrahydrocannabinol (8,9-di-OH-CBT-C5), cannabidiolic A (CBDA C-59-OH-CBT-C5 ester), (−)-(6aR,9S,10S,10aR)-9,10-dihydroxy-hexahydrocannabinol, Cannabiripsol Cannabiripsol-C5, (−)-6a,7,10a-trihydroxy-Δ⁹-tetrahydrocannabinol ((−)-Cannabitetrol), 10-oxo-Δ6a (10a) tetrahydrocannabinol (OTHC); Cannabielsoin-like (CBE): (5aS,6S, 9R, 9aR)-C5-Cannabielsoin (CBEC-5), (5aS,6S, 9R, 9aR)-C3-Cannabielsoin (CBE C-3), (5aS,6S, 9R, 9aR)-Cannabielsoinsäure A (CBEA-C5A), (5aS,6S,9R,9aR)-Cannabielsoinsäure B (CBEA-C5B), (5aS,6S, 9R, 9aR)-C3-Cannabielsoinsäure B (CBEA-C3B), Cannabiglendol-C3 (OH-iso-HI-ICV C-3), Dehydrocannabifuran (DCBF C-5), Cannabifuran (CBF-C5); Isocannabinoide: (−)-Δ⁷-trans-(1R,3R,6R)-Isotetrahydrocannabinol,(±)-Δ7-1,2-cis-(1R,3R,6S/1S,3S,6R)-Isotetrahydro-cannabivarin, (−)-Δ⁷-trans-(1R,3R,6R)-Isotetrahydrocannabivarin; (±)-(1aS,3aR,8bR,8Cr-cannabicyclol (CBL-C5), (±)-(1aS,3aR,8bR,8Cr-Cannabicyclolsäure A (CBLA-C5A) (±)-(1aS,3aR,8bR,8Cr-Cannabicyclovarin (CBLV C-3); Cannabicitran-type (CBT): Cannabicitran (CBT-C5); Cannabichromanon (CBCN C-5), Cannabichromanon-C3 (CBCN C-3), or Cannabicoumaronon (CBCON C-5).

3. The composition of claim 1, wherein the at least one purified cannabinoid is cannabidiol (CBD).

4. The composition according to claim 1, wherein the at least one purified cannabinoid is present in the composition in an amount ranging from about 1 wt % to about 5 wt %.

5.-7. (canceled)

8. The composition according to claim 1, wherein the at least one functional fragrance comprises one or more essential oils.

9. The composition of claim 8, wherein the one or more essential oils comprises one or more of: Zingiber officinale (ginger) root extract; Cinnamomum camphora (camphor) oil; Lavandula angustifolia (lavender) oil; Ormenis multicaulis (chamomile) flower extract; Pogostemon cablin (patchouli) oil; vetiveria zizanoides root (vetivert) oil; Boswellia carterii (frankincense) gum oil; Cinnamomum camphora leaf var linalool (ho wood) oil; citrus aurantium amara (bitter orange) leaf oil; Cymbopogon martini (palmarosa) oil; Eucalyptus globulus leaf oil; Vitis vinifera seed (grapeseed) oil; Chamaemelum nobile flower (Roman chamomile) oil; Cocos nucifera (coconut) oil; Prunus amygdalus dulcis (sweet almond) oil; Simmondsia chinensis (jojoba) seed oil; marjoram oil; black pepper oil; clove oil and combinations thereof.

10. The composition of claim 9, wherein the one or more essential oils comprises lavender, vetivert, and chamomile oil.

11. The composition of claim 10, wherein the one or more essential oil further comprises patchouli oil.

12. The composition of claim 10, wherein the one or more essential oil further comprises ho wood oil.

13. The composition of claim 10, wherein the one or more essential oil further comprises grapeseed oil and/or coconut oil.

14. The composition of claim 10, wherein the one or more essential oil further comprises marjoram oil, black pepper oil and clove oil.

15. The composition of claim 1, wherein the functional fragrance comprises at least one purified terpene.

16. The composition of claim 15, wherein the at least one purified terpene is selected from: linalool, linalool oxide, limonene, geraniol, citronellol, eugenol, citral, farnesol, bakuchiol, derivatives thereof, and combinations thereof.

17. The composition of claim 1, wherein the functional fragrance comprises one or more of the following: Linalool, Linalyl Acetate, Vetiverols, Ocimene, Caryophyllene, Terpentine-4-ol, beta-Pinene, d-Limonene, 1-Octen-3-yl acetate, Geraniol, derivatives thereof, and combinations thereof.

18. The composition of claim 1, further comprising one or more vitamins.

19. The composition of claim 18, wherein the one or more vitamins is Vitamin A.

20. The composition of claim 1, further comprising magnesium chloride and/or Zingiber officinale (ginger) root extract.

21.-30. (canceled)

31. The composition according to claim 1, wherein the composition is in the form of an ointment, a cream, a suspension, a liniment, a balm, a serum, a lotion, a paste, a gel, a spray, an aerosol, a foam, an oil, a liquid, a powder, a patch, a matrix, or a wax.

32. The composition according to claim 31, wherein the composition is for administration by inhalation, nasally, or topically.

33. The composition according to claim 1, wherein the composition is in a sprayable form.

34.-43. (canceled)
